(19) United States
(12) Patent Application Publication
(10) Pub. No.: US 2006/0019393 A1
(43) Pub. Date: Jan. 26, 2006

(54) Minimal lentiviral vector system

(76) Inventors: Paula M. Cannon, South Pasadena, CA (US); Celina Ngiam, Los Angeles, CA (US)

Correspondence Address: MYERS BIGEL SIBLEY & SAJOVEC, PO BOX 37428, RALEIGH, NC, 27627, US

(21) Appl. No.: 11/003,967

(22) Filed: Dec. 3, 2004

Related U.S. Application Data
(60) Provisional application No. 60/526,668, filed on Dec. 4, 2003.

(51) Int. Cl. C12N 15/867

(52) U.S. Cl. 435/456

(57) ABSTRACT

A lentiviral vector system is described. The system comprises a lentiviral transfer vector and a packaging construct. The transfer vector comprises (a) a 5′ LTR; (b) a 3′ LTR comprising a polyadenylation signal; (c) a minimal packaging signal, (d) (i) at least one heterologous upstream enhancer (UE) sequences, and/or (ii) at least one additional copy of endogenous UE sequences operatively associated with said polyadenylation signal; and (e) a PRE. The packaging construct comprises a nucleic acid encoding and expressing a lentiviral Gag nucleic acid (preferably Gag and Pol). Preferably the lentiviral Gag nucleic acid is a mutated Gag nucleic acid containing one or more substitution mutations, wherein said mutated Gag nucleic acid encodes the same amino acid sequence as the corresponding unmutated Gag nucleic acid, but differs from the nucleic acid sequence of said corresponding unmutated Gag nucleic acid sequence due to the degeneracy of the genetic code. Preferably the packaging construct further comprises a heterologous nucleic acid encoding and expressing an adenovirus VA RNA. The transfer vector and packaging construct can be used together in a producer cell to produce viral particles.

### RELATED APPLICATIONS

This application claims the benefit of U.S. Provisional Patent Application Ser. No. 60/526,668, filed Dec. 4, 2003, the disclosure of which is incorporated by reference herein in its entirety.

### FIELD OF THE INVENTION

The present invention concerns lentiviral vectors, methods and constructs for making the same, and methods of using the same.

### BACKGROUND OF THE INVENTION

Lentiviral vectors offer compelling advantages for many gene therapy applications because (1) their ability to transduce non-dividing cells allows for gene transfer to primary cells with minimal manipulation in vitro, and (2) they can permanently integrate into a host cell genome, thereby maintaining vector gene expression as cells divide. These features make the vectors especially suited to gene therapy applications which require efficient transduction of relatively quiescent cells and the long-term expression of the vector gene in these cells and their progeny.

Several improvements have been made to the design of lentiviral vectors since they were first described (Naldini L, et al., 1996. In vivo gene delivery and stable transduction of nondividing cells by a lentiviral vector. Science 272:263-7). These initial vectors retained large stretches of the HIV-1 genome, but later versions reduced the extent of HIV-1 sequences. Improvements to date include the following:

(1) Self-inactivating (SIN) vectors are now commonly used; this modification involves a large deletion within the U3 region of the LTR that effectively removes any residual promoter activity and thus allows the use of an alternate internal promoter to drive transgene expression (eg Zufferey R, et al., 1999. Woodchuck hepatitis virus posttranscriptional regulatory element enhances expression of transgenes delivered by retroviral vectors. J. Virol. 73:2886-92). Together with the modification of the packaging plasmid to replace the LTR promoter with heterologous promoters (eg CMV), this also allowed elimination of Tat from the system (Dull T, et al., 1998. A third-generation lentivirus vector with a conditional packaging system. J. Virol. 72:8463-71).

(2) Removal of the HIV-1 “accessory proteins” nef, vif, vpu and vpr from the packaging plasmid (Zufferey R, et al., 1997. Multiply attenuated lentiviral vector achieves efficient gene delivery in vivo. Nature Biotech. 15:871-75)

(3) Placement of Rev onto a separate plasmid, so making a 4-plasmid system (“third generation vectors”) (Dull 1998, supra).

Despite the reduction in HIV sequences, regions of overlap still remain between the transfer vector and the packaging component.

In addition, since lentiviral vectors are made by transient transfection which significantly increases the likelihood that recombination events will occur, the risk of both homologous and non-homologous recombination during transfection procedures is high (Mann R, et al., 1983. Construction of a retrovirus packaging mutant and its use to produce helper-free defective retrovirus. Cell 33:153-9).

The proposed use of any viral-derived vector system for human gene therapy raises concerns about the generation of replication-competent viruses. Such variants could have immediate pathogenic consequences for the patient, could evolve over time to become more pathogenic, or could be mobilized and spread by co-infections with a wild-type virus (HIV-1). The viruses so generated could also be transmitted to other individuals. For a vector system derived from a human pathogen such as HIV-1, these concerns are naturally increased. Accordingly, there remains a need for new approaches to the production of lentiviral vectors.

### SUMMARY OF THE INVENTION

In order to reduce the likelihood of recombination among the vector components, or between transfer vectors and HIV-1, we here describe a minimal lentiviral vector system in which we have eliminated overlap of homologous HIV-1 sequences between the three plasmid components that contain HIV-1 sequences. We believe these modifications significantly reduce the risk for generation of repliction competent lentivirus (RCL). In addition, we describe a set of transfer vectors that can be used with this system. Starting with the basic “non-overlapping” vector, SM, we describe improvements in vector performance due to the addition of various cassettes.

Thus, a first aspect of the present invention is a retroviral or retroviral or lentiviral transfer vector comprising:
a) a 5′ LTR;
b) a 3′ LTR comprising a polyadenylation signal;
c) a minimal packaging signal,
d) (i) at least one heterologous upstream enhancer (UE) sequences, and/or (ii) at least one additional copy of endogenous UE sequences operatively associated with the polyadenylation signal;
e) a PRE (for example, a WPRE); and
f) optionally, but in some embodiments preferably, a cPPT.

In some embodiments the vector comprises several heterologous UE sequences at least two of which are derived from the same UE. In some emodiments the vector comprises several heterologous UE sequences at least two of which are derived from different UEs; several additional copies of endogenous UE sequences at least two of which copies have identical sequences; and/or several additional copies of endogenous UE sequences at least two of which copies have different sequences.

In some embodiments of the vector, the 3′ LTR comprises a deletion of U3 promoter sequence. In some emdoiments the heterologous UE sequence, or at least one of the heterologous UE sequences, or the additional copy of endogenous UE sequence, or at least one of the additional copies of endogenous sequences is at the U3 promoter deletion site.

In some embodiments of the vector, the heterologous UE sequence or at least one of the heterologous UE sequences is a viral or animal UE sequence; In some emdoiments of the vector the 3′ LTR comprises an endogenous UE sequence.

In some embodiments of the vector, the 5′ LTR comprises a heterologous promoter inserted in the U3 region. In some embodiments of the vector, the 5′ LTR comprises a deletion of the endogenous U3 promoter sequence.

In some embodiments, the vector comprises a heterologous coding sequence which is 3′ downstream of the 5′ LTR and 5′ upstream of the 3′ LTR. In some embodiments the the heterologous coding sequence encodes a protein, peptide or RNA; in other embodiments the heterologous coding sequence encodes a negative selective marker.

A particular embodiment of the vector is one having the nucleic acid sequence given herein as SEQ ID NO: 20, subject to the proviso that a heterologous coding sequence may optionally be inserted therein 3′ downstream of the vector 5′ LTR and 5′ upstream of the vector 3′ LTR.

A second aspect of the invention is cell comprising or containing the transfer vector described above. In some embodiments the cell is a mammalian cell, such as a human cell.

A third aspect of the invention is a cell comprising or containing a proviral sequence transcribed from the vector described. Again, in some embodiments the cell is a mammalian cell, such as a human cell.

A fourth aspect of the invention is an infectious retroviral or lentiviral particle produced by a cell as described above, wherein the cell is a producer cell (as discussed further below).

A fifth aspect of the invention is a method for expressing a heterologous coding sequence in a cell comprising delivering to the cell (e.g., transfecting, inserting, electroporating, etc.) the transfer vector or infectious viral particle as described above, the transfer vector, infectious viral particle or encoding and expressing the heterologous coding sequence in the cell into which it is delivered. In some embodiments the cell is in a subject.

A further aspect of the invention is a method for expressing a heterologous coding sequence in a subject comprising administering or transfusing the subject with a composition comprising cells as described above. Subjects may be mammalian subjects, including human subjects.

A further aspect of the invention is a packaging construct comprising a nucleic acid encoding and expressing a retroviral or lentiviral Gag nucleic acid; wherein the retroviral or lentiviral Gag nucleic acid is a mutated Gag nucleic acid containing one or more substitution mutations, wherein the mutated Gag nucleic acid encodes the same amino acid sequence as the corresponding unmutated Gag nucleic acid, but differs from the nucleic acid sequence of the corresponding unmutated Gag nucleic acid sequence due to the degeneracy of the genetic code. Preferably the packaging construct is oneencoding and expressing retroviral or lentiviral Gag and Pol nucleic acid.

In some embodiments of the packaging construct, the first 30 or 40 5′ nucleic acids of the mutated Gag nucleic acid do not contain more than 12 consecutive unmutated nucleic acid residues.

In some embodiments the packaging construct further comprises a heterologous nucleic acid encoding and expressing an adenovirus VA RNA. (e.g., adenovirus VA1 RNA, adenovirus VA2 RNA, or combinations thereof.

In a particularly preferred embodiment the packaging construct has the sequence given herein as SEQ ID NO: 26.

Another aspect of the invention is a retroviral or lentiviral vector producer cell comprising a heterologous nucleic acid encoding and expressing a retroviral or lentiviral Gag nucleic acid; wherein the retroviral or lentiviral Gag nucleic acid is a mutated Gag nucleic acid containing one or more substitution mutations, wherein the mutated Gag nucleic acid encodes the same amino acid sequence as the corresponding unmutated Gag nucleic acid, but differs from the nucleic acid sequence of the corresponding unmutated Gag nucleic acid sequence due to the degeneracy of the genetic code. Preferably, the heterologous nucleic acid encodes and expresses retroviral or lentiviral Gag and Pol nucleic acid. Preferably, the first 30 or 40 5′ nucleic acids of the mutated Gag nucleic acid do not contain more than 12 consecutive unmutated nucleic acid residues. Preferably the producer cell further comprises a heterologous nucleic acid encoding and expressing an adenovirus VA RNA (e.g., adenovirus VA1 RNA, adenovirus VA2 RNA, or combinations thereof.

A further aspect of the invention is a method of making retroviral or lentiviral particles comprising the steps of: (a) providing a retroviral or lentiviral vector producer cell (e.g., a mammalian cell) comprising a heterologous nucleic acid encoding and expressing a retroviral or lentiviral Gag nucleic acid; wherein the retroviral or lentiviral Gag nucleic acid is a mutated Gag nucleic acid containing one or more substitution mutations, wherein the mutated Gag nucleic acid encodes the same amino acid sequence as the corresponding unmutated Gag nucleic acid, but differs from the nucleic acid sequence of the corresponding unmutated Gag nucleic acid sequence due to the degeneracy of the genetic code; (b) introducing the retroviral or lentiviral vector described above into the producer cell, the retroviral or lentiviral vector further comprising the corresponding unmutated Gag nucleic acid; and then (c) collecting retroviral or lentiviral particles from the producer cells. Preferably, the heterologous nucleic acid in the producer cell encodes and expresses retroviral or lentiviral Gag and Pol nucleic acid.

A further aspect of the invention is a packaging construct comprising: a nucleic acid encoding and expressing a retroviral or lentiviral Gag nucleic acid; and a heterologous nucleic acid encoding and expressing an adenovirus VA RNA. Preferably the nucleic acid encodes and expresses retroviral or lentiviral Gag and Pol nucleic acid. The adenovirus VA RNA may be adenovirus VA1 RNA, adenovirus VA2 RNA, or combinations thereof.

A further aspect of the invention is a retroviral or lentiviral vector producer cell comprising: a heterologous nucleic acid encoding and expressing a retroviral or lentiviral Gag nucleic acid; and a heterologous nucleic acid encoding and expressing an adenovirus VA RNA. Preferably the heterologous nucleic acid encodes and expresses retroviral or lentiviral Gag and Pol nucleic acid. The adenovirus VA RNA may be adenovirus VA1 RNA, adenovirus VA2 RNA, or combinations thereof.

A further aspect of the invention is a method of making retroviral or lentiviral particles comprising the steps of: (a) providing a retroviral or lentiviral vector producer cell (e.g., a mammalian cell) comprising or containing a heterologous nucleic acid encoding and expressing a retroviral or lentiviral Gag nucleic acid, and a heterologous nucleic acid encoding and expressing an adenovirus VA RNA; (b) introducing the retroviral or lentiviral vector described above into the producer cell; and then (c) collecting retroviral or lentiviral particles from the producer cells. 62. The method of claim 61, wherein the producer cell is a mammalian cell. Preferably the heterologous nucleic acid in the producer cell encodes and expresses retroviral or lentiviral Gag and Pol nucleic acid.

The present invention is explained in greater detail in the specification set forth below.

### BRIEF DESCRIPTION OF THE DRAWINGS

FIG. 1 shows the main features and cloning sites of the lentiviral transfer vector pIC (not drawn to scale).

FIG. 2 shows fragment 1 from which the lentiviral transfer vector pIC was assembled.

FIG. 3 shows fragment 2 from which the lentiviral transfer vector pIC was assembled.

FIG. 4 shows fragment 3 from which the lentiviral transfer vector pIC was assembled.

FIG. 5 shows fragment 4 from which the lentiviral transfer vector pIC was assembled.

FIG. 6 shows fragment 5 from which the lentiviral transfer vector pIC was assembled.

FIG. 7 shows fragment 6 from which the lentiviral transfer vector pIC was assembled.

FIG. 8 shows fragment 7 from which the lentiviral transfer vector pIC was assembled.

FIG. 9 shows the final sequence (SEQ ID NO: 20) of the lentiviral transfer vector pIC, when assembled.

FIG. 10. Codon alterations in first 40 bp of Gag open-reading frame to reduce homology between pGPRcw and lentiviral transfer vectors.

FIG. 11 shows the final sequence of the packaging construct pGPRcwVA, when assembled (SEQ ID NO: 26).

FIG. 12 shows the effect on titer of the presence of various elements in transfer vector pIC.

### DETAILED DESCRIPTION OF THE PREFERRED EMBODIMENTS

Unless otherwise specified herein, the following words and terms shall have the following meanings with respect to the present disclosure and the appended claims.

“3′ LTR” refers to a 3′ retroviral or lentiviral long terminal repeat, which may or may not be modified from its corresponding native (i.e., that existing in the wild-type retrovirus) 3′ LTR by deleting and/or mutating endogenous sequences and/or adding heterologous sequences. The 3′ LTR may be natural or synthetic.

“5′ LTR” refers to a 5′ retroviral or lentiviral long terminal repeat, which may or may not be modified from its corresponding native 5′ LTR by deleting and/or mutating endogenous sequences and/or adding heterologous sequences. The 5′ LTR may be natural or synthetic.

“3′ LTR polyadenylation signal” refers to the polyadenylation signal present in the 3′ LTR of retroviruses or lentiviruses. The polyadenylation signal may be natural or synthetic.

“Upstream enhancer” and “UE” are used interchangeably, and refer to a control element present in the 3′ untranslated region of various eukaryotic and viral genes that enhances transcriptional termination by a polyadenylation signal located downstream of the enhancer. Examples of UEs are found in the SV40 late polyadenylation signal (USE), the HIV-1 LTR (UHE) and the ground squirrel hepatitits virus (UGE). The UEs may be natural or synthetic.

“Upstream enhancer sequence” and “UE sequence” are used interchangeably, and refer to the sequence of a UE or an active segment thereof. Like a UE, an active segment of a UE increases the transcriptional termination activity of a polyadenylation signal when it is placed 5′ upstream of that signal. A UE may comprise many active segments that may or may not be overlapping in sequence.

In the context of the viral vectors of the invention, a “heterologous” UE sequence is a UE sequence from a UE not identical to the one present in the native 3′ LTR of the virus from which the viral vector of the invention is derived. By contrast, an “endogenous” UE sequence is a UE sequence from a UE present, such as UHE of HIV-1, in the native 3′ LTR of the virus from which the viral vector of the invention is derived.

“3′ transcription termination structures” of a LENTIviral vector refer to structures within and proximal to the 3′ LTR that effect termination of transcriptions initiated upstream of the structures. Such structures comprise the 3′ LTR polyadenylation signal and may additionally comprise endogenous UE sequences and heterologous UE sequences operatively associated with that signal. The structures may be natural or synthetic.

“Polynucleotide” refers interchangeably to natural or synthetic double- and single-stranded molecules. Unless otherwise specified or required, any embodiment of the invention described herein that is a polynucleotide encompasses both the double-stranded form and each of two complementary single-stranded forms known or predicted to make up the double-stranded form.

“Retrovirus” denotes a class of viruses which use RNA-directed DNA polymerase, or “reverse transcriptase” to copy a viral RNA genome into a double-stranded DNA intermediate which integrates into the chromosomal DNA of a host cell. Retroviruses include lentiviruses. Examples of retroviruses include, but are not limited to, Moloney murine leukemia virus, spleen necrosis virus, Rous sarcoma virus, Harvey sarcoma virus, avian leukosis virus, human immunodeficiency virus, myeloproliferative sarcoma virus, and mammary tumour virus.

“Lentivirus” denotes a category of retroviruses particularly preferred for the present invention. Examples of lentiviruses include human immunodeficiency virus, simian immunodeficiency virus, equine infectious anemia virus, feline immunodeficiency virus, visna virus.

“Viral vector genome” refers to a polynucleotide comprising sequences from a viral genome that are sufficient to allow an RNA version of that polynucleotide to be packaged into a viral particle, and for that packaged RNA polynucleotide to be reverse transcribed and integrated into a host cell chromosome by the action of the viral enzymes, such as reverse transcriptase and integrase, contained in the viral particle.

“Gene” refers to a polynucleotide that encodes a polypeptide.

“Coding sequence” refers to a polynucleotide that encodes a polypeptide, antisense RNA, a ribozyme, a small interfering RNA, or a structural RNA, such as snRNA, tRNA and rRNA.

In the context of the viral vectors of the invention, a “heterologous” gene or coding sequence is a gene or coding sequence that is not identical to any gene or coding sequence found in the virus from which the viral vector of the invention is derived.

Two genes or sequences are “identical” if the order of nucleotides in each gene or sequence is the same, without any addition, deletion or material substitution.

In the context of polynucleotides, a “sequence” is an order of nucleotides in a polynucleotide in a 5′ to 3′ direction in which residues that neighbor each other in the sequence are contiguous in the primary structure of the polynucleotide.

“Operatively associated” refers to a juxtaposition of genetic elements, wherein the elements are in a relationship permitting them to operate in the expected manner. For example, a UE sequence is operatively linked to a polyadenylation signal in the same DNA molecule if the UE sequence enhances transcriptional termination by that signal. Similarly, a promoter is operatively associated with a coding region in the same DNA molecule if the promoter enables transcription of the coding sequence. There may be intervening residues between such associated elements so long as their functional relationship is maintained.

“cPPT” as used herein refers to a sequence termed the central polypurine tract.

“PRE” as used herein refers to cis-acting posttranscriptional regulatory elements such as WPREs.

“WPRE” as used herein refers to the woodchuck hepatitis b virus post-transcriptional regulatory element.

“Packaging construct” as used herein refers to a nucleic acid containing (and in a producer cell expressing) at least lentiviral or retroviral Gag, preferably expressing both lentiviral or retroviral Gag and Pol, and optionally also containing (and in a producer cell expressing) additional lentiviral genes, including but not limited to Tat, Rev, Vif, Vpu, Vpr, Nef and Env.

“Producer cell” as used herein refers to (i) cells that stably and constitutively express the proteins required for packaging of vector particles, (ii) cells that stably and inducibly express the proteins required for packaging of vector particles, and (iii) transiently transfected cells that express the proteins required for packaging of the vector particles for a limited period of time.

“Minimal packaging signal” as used herein refers to a packaging signal in a transfer vector construct that has been reduced in size (e.g., to include not more than 40, 60, or 80 continuous nucleic acids of the gag gene) and/or a packaging signal in a transfer vector construct that contains one or more substitution mutations therein to reduce or minimize homology to a corresponding portions of the GAG ORF in separate nucleic acid construct(s) in the producer cell, in like manner as described with respect to mutated gag sequences for packaging constructs below.

Subjects that may be treated by the methods described herein may be human subjects, or other mammalian subjects such as dogs, cats, mice, goats, sheep, cattle, pigs, etc. for veterinary purposes or for the production of biological materials in those subjects.

The disclosures of all United States patents cited herein are to be incorporated by reference herein in their entirety.

In general, the assembly of various elements into lentiviral vectors, the assembly of such elements into producer cells, the production viral particles from such vectors and producer cells, and methods of use of such particles, can be carried out in accordance with known techniques, including but not limited to those described in U.S. Pat. No. 6,808,923 to Engelman et al.; U.S. Pat. No. 6,808,905 to McArthur et al.; U.S. Pat. No. 6,620,595 to Cannon et al.; U.S. Pat. No. 6,797,512 to McGuinness et al.; U.S. Pat. No. 6,428,953 to Naldini et al.; U.S. Pat. No. 6,218,181 to Verma et al.; and U.S. Pat. No. 6,143,520 to Marasco et al.

1. Lentiviral Vectors.

In general, the vectors of the invention comprise a viral vector genome having a 5′ LTR, a 3′ LTR comprising a polyadenylation signal, and a packaging signal. The viral vector genome may be from a lentivirus including, but not limited to, human immunodeficiency virus, simian immunodeficiency virus, equine infectious anemia virus, feline immunodeficiency virus, visna virus. Preferably, the vectors comprise a viral vector genome that is replication defective. Typically, such a defect is due to a mutation and/or deletion of one or more viral structural and replication functions (e.g., gag, pol, env). Accordingly, vectors of the invention may be derived from replication defective viral vectors in the art as noted below.

More preferably, vectors of the invention are derived from replication defective lentiviral vectors including, but not limited to, those based on: HIV-1, such as pHR′CMVlacZ, pHR′CMVIacZ SIN18, and pRRLPGK-GFP (Zufferey et al., J. Virol. 72:9873-9880 (1998)), LL-CG, CL-CG, LS-CG, CS-CG and CL-G (Miyoshi et al., J. Virol. 72. 8150-8157 (1998)), pV653CMVβ-gal (Gasmi et al., J Virol 73:1828-1834 (1999)), pTV (Iwakuma et al., Virology 261:120-132 (1999)), pH3Z, pH4Z, pH5Z (Kim et al.,. Virol. 72:811-816 (1998)); HIV-2 (Arya et al., Hum Gene Ther.9:1371-80 (1998)); simian immunodeficiency virus, such as pVG (Schnell et al., Hum Gene Ther 11:439-47 (2000)); feline immunodeficiency virus, such as FIV-gal (Wang et al., J Clin Invest.104:R55-62 (1999)), PTFIV (Johnston et al., J Virol 73:4991-5000 (1999)); equine infectious anemia virus, such as pONY2.101acZ, pONY4.0Z (Mitrophanous et al., Gene Ther. 6:1808-18 (1999)).

Most preferably, the vectors of the invention are derived from SIN lentiviral vectors, such as pHR′CMVlacZ SIN18 (Zufferey et al., J. Virol. 72:9873-9880 (1988)), LS-CG, CS-CG (Miyoshi et al., J. Virol. 72. 8150-8157 (1988)), SIN-W-PGK (Deglon et al., Hum Gene Ther. 11:179-190 (2000)), pVG (Schnell et al., Hum Gene Ther. 11:439-47 (2000)), and pTV (Iwakuma et al., Virology 261:120-132 (1999)). Particularly preferred is the SMPU vector described in M. Chen M et al., Restoration of Type VII Collagen Expression and Function In Dystrophic Epidermolysis Bullosa. Nat. Genet. 32:670-675, 2002 (also described in U.S. Pat. No. 6,620,595 to Cannon et al.).

The 5′ LTR of the vectors may be an unmodified viral 5′ LTR. That is, a native 5′ LTR as it exists in a lentivirus or retrovirus. In a preferred embodiment, the endogenous U3 promoter of the 5′ LTR has been inactivated by mutation and/or deletion and replaced with a heterologous promoter. The activity of the heterologous promoter may be constitutive, inducible or target cell-specific (i.e., expression is preferential or limited to one or several specific cell types and not or less so in other cell types). Useful heterologous promoters include, but are not limited to, CMV (Miyoshi et al., J. Virol. 72. 8150-8157 (1988)), Rous sarcoma virus promoter (Dull et al., J. Virol. 72:8463-71 (1998)), tetracycline-inducible promoter (Hwang et al., J. Virol. 71:7128-31 (1997)).

The 3′ LTR of the vectors may be an unmodified lentiviral or retroviral 3′ LTR. In a preferred embodiment, the endogenous U3 promoter of the 3′ LTR has been inactivated by mutation or deletion. In a more preferred embodiment, such inactivation is specific to the U3 promoter. That is, the inactivation does not adversely affect other structures, such as the att sequence needed for integration and any endogenous UE, of the 3′ LTR. In a most preferred embodiment, the promoter is inactivated by mutating or deleting sequences in the region that corresponds to about residues 9113 to 9506 of the pNL4-3 strain of HIV-1.

The vectors of the invention have an enhanced 3′ transcription termination structure, which may comprise one or several UE sequences operably associated with the polyadenylation signal in the 3′ LTR. The UE sequence may be a heterologous UE sequence or an additional copy of any endogenous UE sequence which may be present in the 3′ LTR. In one embodiment, the 3′ transcription termination structure comprises one or several heterologous UE sequences. In another embodiment, the 3′ transcription termination structure comprises one or several additional copies of an endogenous UE sequence. In a future embodiment, the 3′ transcription termination structure comprises both heterologous and an additional copy of endogenous UE sequences.

The vectors of the invention may additionally comprise a microbial origin of replication and a microbial screenable or selectable marker for use in amplifying vector sequences in microbial cells, such as bacteria and yeast.

Upstream enhancers. The vectors of the invention may comprise any UE, as described in greater detail in U.S. Pat. No. 6,620,595 to Cannon et al. Preferably, the UE is from an eukaryotic or viral gene. Example viral UEs include, but are not limited to, those of SV40 virus (e.g., USE), cauliflower mosaic virus, HIV-1 (e.g., UHE), ground squirrel hepatitis virus (e.g. UGE), or equine infectious anemia virus UE. Example of eukaryotic UEs include, but are not limited to, those of mammalian complement C2 and lamin B2 genes. In preferred embodiments, the retroviral vectors comprise the USE from SV40 or the UGE from ground squirrel hepatitits virus.

The transcriptional termination structures used in the construct may comprise the 3′ untranslated region of an eukaryotic or viral gene. Preferably, the 3′ untranslated region comprises an endogenous polyadenylation signal. In one embodiment, the transcription termination structure comprises a viral 3′ LTR. In a preferred embodiment, the transcription termination structure comprises a modified 3′ LTR, wherein the U3 promoter is inactivated by deletion or other means. In a more preferred embodiment, the transcription termination structure comprises a 3′ LTR that is to be incorporated into a vector of the invention.

The UE segment may be inserted anywhere in the region between the coding sequence of the first reporter polypeptide and the polyadenylation signal of the first transcription termination structure. Preferably, the segment is inserted less than 100 nucleotides upstream of the polyadenylation signal. More preferably, the segment is inserted less than 50 nucleotides upstream of the polyadenylation signal. Most preferably, the segment is inserted less than 20 nucleotides upstream of the polyadenylation signal.

Specific embodiments of UEs and active UE segments (i.e., UE sequences collectively) that may comprise vectors of the invention include, but are not limited to, the following: a) The UE from SV40 (USE); b) The equine infectious anemia virus UE; c) The cauliflower mosaic virus UE comprising the sequence; d) The ground squirrel hepatitis virus UE (UGE); e) The adenovirus L3 UE comprising the sequence; f) The HIV-1 UE (also known as UHE); g) The complement C2 UE; h) The lamin B2 UE; and g) active fragments of the foregoing (see U.S. Pat. No. 6,620,595 to Cannon et al.).

The vectors of the invention comprise one or several UE sequences that are operatively associated with the 3′ LTR polyadenylation signal. Specifically, the operative association refers to an incorporation of UE sequence(s) that enhances transcriptional termination activity of the vector 3′ LTR. Vectors having enhanced transcription termination may have various improved properties. Possible improvements include reduced transcription read-through into flanking vector or host sequences; increased production of vector RNA and/or vector encoded polypeptide; and higher vector titers in producer cells. According to the invention, a UE sequence is operatively associated with the 3′ LTR polyadenylation signal if the incorporation effects more than about 10% improvement in any of these properties.

A UE sequence may be operatively associated with the 3′ LTR polyadenylation signal by inserting the sequence at a vector site that is 5′ upstream of the signal. The orientation of the inserted UE sequence to the polyadenylation signal should be same as its orientation to the polyadenylation signal in the gene from which the sequence was derived. Preferably, the insertion site is less than about 500 nucleotides upstream of the signal, using the unmodified vector as a reference. More preferably, the site is less than about 100 nucleotides upstream. Even more preferably, the site is less than about 50 nucleotides upstream. In a particularly preferred embodiment, the insertion is at a site in the U3 region of the 3′ LTR. In a further preferred embodiment, such site is between the att sequence and any endogenous UE sequence that may be present in the U3 region. In an even more preferred embodiment, a 3′ LTR sequence between the att sequence and the R region containing some or all of the U3 promoter structure is deleted and the UE sequence is inserted at the deletion site. In a most preferred embodiment, such deletion span the region that corresponds to about residues 9113 to 9506 of the pNL4-3 strain of HIV-1 and the UE sequence is inserted into the deletion site.

Vectors Comprising a Plurality of UE Sequences. The vectors of the invention may comprise a plurality of UE sequences which are operably associated with the 3′ LTR polyadenylation signal. The invention contemplates vectors comprising all possible combinations of multiple UE sequences. Example combinations include, but are not limited to: two or more heterologous UE sequences are identical or are derived from the same UE; two or more heterologous UE sequences that are derived from different UEs; two or more copies of the same endogenous UE sequence, two or more copies of different endogenous UE sequences; one or more heterologous UE sequence and one or more additional copies of an endogenous UE sequence.

Postranscriptional regulatory elements. The vectors of the present invention preferably include one or more posttranscriptional regulatory element. PREs such as the WPRE are known and described in, for example, U.S. Pat. Nos. 6,312,912 and 6,287,814, which disclose posttranscriptional regulatory elements useful for efficient RNA export of RNA is provided. WPRE was originally derived from woodchuck hepatitis virus. Other PREs (sometimes also termed “RNA export elements” are described in U.S. Pat. No. 6,677,500, and include, but are not limited, to Mertz sequences (also described in U.S. Pat. Nos. 5,914,267 and 5,686,120). PREs and particularly WPREs are further described in U.S. Pat. Nos. 6,555,342; 6,312,912; 6,287,814; 6,808,905; 6,800,281; and 6,712,612.

Central polypurine tract elements. In some embodiments of the present invention, the vectors include one or more central polypurine tract element, or “cPPT”. cPPT elements are known and described in, for example, U.S. Pat. Nos. 6,800,281; 6,649,159; 6,627,442; and 6,682,907. As noted in U.S. Pat. No. 6,682,907, the identification of cPPT sequences is facilitated by the fact that a polypurine sequence located at the upstream edge (5′) of the 3′ LTR in all retroviruses is repeated in the center of the genome in lentiviruses. This cPPT sequence can be an exact repeat as in the HIV-1 virus, or slightly modified in other lentivirus. If necessary, the sequence of nucleotides comprising cPPT can be point mutated or mutated by deleting or inserting nucleotides. By way of example, point mutations have been produced in the cPPT sequence of HIV-1 and have shown that it retained residual infectivity in the cells (Chameau et al, J. Virol. 1992, 66, p 2814-2820).

Heterologous Genes and Coding Sequences. The vectors of the invention may be beneficially used to express desired gene products in mammalian cells and organisms. Accordingly, the vectors may additionally comprise one or more heterologous coding sequences, wherein such sequences are derived from sources other than the retroviral genome from which the vectors are derived.

In one embodiment, the heterologous coding sequences are inserted into the viral backbone, preferably between the 5′ and 3′ LTRs, such that they are operably associated with the 5′ LTR promoter. Where such insertions lead to production of polycistronic mRNA comprising the heterologous coding sequences, it may be advantageous to also operatively associate each heterologous coding sequence with an IRES in order to achieve efficient translation of each sequence.

In another embodiment, the heterologous coding sequences are each operably associated with an individual promoter to form expression constructs, and such constructs are into the viral backbones, preferably between the 5′ and 3′ LTRs. The expression constructs may comprise promoters that are constitutive, inducible, tissue-specific, or cell-cycle specific. Examples of useful promoters include, but are not limited to, the SV40 promoter, CMV promoter, adenovirus promoters, B19 parvovirus promoters, histone promoter, pol III promoter, and beta-actin promoters.

Diverse gene products may be expressed using vectors of the invention. They include polypeptides, proteins and RNAs such as structural RNAs, anti-sense RNAs, short interfering RNAs, and ribozymes. In one embodiment, the vectors of the invention comprise and express one or more heterologous sequences encoding therapeutic polypeptides. Example therapeutic polypeptides include cytokines, growth factors, hormones, kinases, receptors, receptor ligands, enzymes, antibody polypeptides, transcription factors, blood factors, and artificial derivatives of any of the foregoing.

In another embodiment, the vectors of the invention comprises and express one or more heterologous sequences encoding negative selectable markers. The negative selectable markers may be cytotoxins that directly or indirectly inhibit or kill a host cell. Examples of “direct” cytotoxins include the active moieties of cholera and botulism toxins. In a preferred embodiment, the vectors of the invention comprise and express one or more heterologous sequences encoding indirect cytotoxins. The indirect cytotoxins by themselves are not toxic but achieve cellular inhibition by interacting with another agent. An example is HSV-thymidine kinase (TK) which is non-toxic but can activate drugs like ganciclovir into a toxic nucleotides that kill mammalian cells.

While the present invention is described primarily with respect to lentiviruses and lentiviral vectors, those skilled in the art will appreciated that the invention may also be implemented as other retroviral vectors in accordance with known techniques.

2. Producer Cells.

A producer cell of the present invention contains and expresses the nucleic acids (typically provided on a packaging construct) not found in the transfer vector, but otherwise necessary for the production of viral particles when the transfer vector is inserted into the producer cell. Producer cells of the invention are, in general, mammalian cells, and in some embodiments are human cells.

A producer cell generally comprises a heterologous nucleic acid encoding and expressing lentiviral Gag and Pol nucleic acids, may also comprise a heterologous nucleic acid encoding and expressing lentiviral Rev nucleic acid, and may also comprise a heterologous nucleic acid encoding and expressing a fusion protein (FP) nucleic acid. Examples of fusion proteins include vesicular stomatitis virus G protein (VSV G), the amphotropic murine leukemia virus Env protein, the retroviral RD 114 Env protein, and the lymphochoriomeningitis virus glycoprotein.

The various nucleic acids may be transiently or stably inserted into the producer cell on a single vector nucleic acid, or multiple vector nucleic acids.

In the present invention, the lentiviral Gag nucleic acid in the producer cell is a mutated Gag nucleic acid containing one or more substitution mutations, wherein said mutated Gag nucleic acid encodes the same amino acid sequence as the corresponding unmutated Gag nucleic acid, but differs from the nucleic acid sequence of said corresponding unmutated Gag nucleic acid sequence due to the degeneracy of the genetic code. Preferably, these mutations are positioned such that the first 30 or 40 5′ nucleic acids of the mutated Gag nucleic acid does not contain more than 12 consecutive unmutated nucleic acid residues, and in some embodiments these mutations are positioned such that the mutated Gag nucleic acid does not contain more than 8 or 10 consecutive unmutated nucleic acid residues. Thus the mutated Gag nucleic acid preferably contains at least 2, 3, 4 or 5 or more such substitution mutations. Additional substitution mutations may be included if desired and substitution mutations may be positioned adjacent to one another if desired. An advantage of the present invention is that, in some embodiments, the substitution mutations may be chosen to increase or maximize codon preference for the mammalian (or in some embodiments human) producer cell, thereby increasing the titers of viral particles produced by such cells.

Nucleic acids encoding Pol in the producer cells may be mutated in like manner as described with nucleic acids encoding Gag as described above.

The packaging constructs and producer cells may be constructed so that the Gag (or more preferably Gag-Pol together), Rev and/or FP are constitutively, inducibly or transiently expressed, in accordance with known techniques.

Adenovirus VA. In some embodiments the producer cells contain and transcribe a nucleic acid encoding adenovirus VA RNA. Nucleic acids encoding adenovirus VA RNA, including adenovirus VA₁ RNA and adenovirus VA₂ RNA, are known and described in, for example, U.S. Pat. Nos. 6,762,038; 6,482,633; 6,004,797; 5,945,335; and 5,837,503. The nucleic acid encoding the adenovirus VA RNA may be on the same or different vector or vectors carrying the other heterologous components inserted into the producer cells.

3. Production of Viral Particles and Uses Thereof.

Infectious iviral particles comprising vectors of the invention may be produced by methods well known in the art. For example, vector particles can be produced by transfecting a packaging cell expressing in trans the required lentiviral replication functions, such as Gag-Pol, Rev and FP proteins. Gag and Pol provide viral structural and enzymatic components, Rev is required to ensure nucleic export and subsequent translation of unmodified HIV-1 Gag-Pol mRNAs containing a Rev response element (RRE), and the FP functions to target vector particles to target cells. FP function can comprise an envelope (Env) protein from any retrovirus or a fusion or spike protein from another enveloped virus (e.g., VSV G protein) or any molecule that binds a specific cell surface receptor.

The infectious vector particles of the invention may be used to express heterologous coding sequences in mammalian cells and organisms in accordance with known techniques. In one embodiment, an effective dose of infectious vector particles comprising a heterologous coding sequence is administered directly to the mammalian organism to achieve transduction of target cells within the organism. In another embodiment, mammalian cells are transduced in vitro with the such vector particles and the transduced cells are then administered in vivo to a host. Preferably, the infectious vector particles of the invention are used to express heterologous coding sequences in primates and primate cells. More preferably, the vector particles of the invention are used to to express heterologous coding sequences in humans and human cells.

The present invention is explained in greater detail in the following non-limiting Examples.

### EXAMPLE 1

### Lentiviral Transfer Vector pIC: General Features

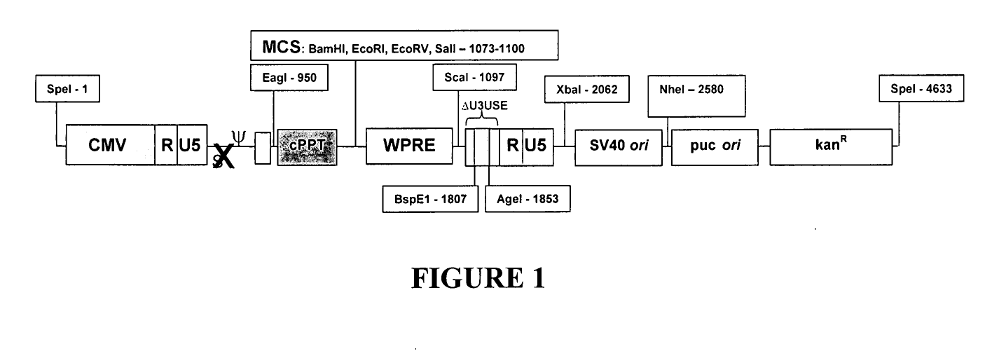

FIG. 1 shows the main features and cloning sites of the lentiviral transfer vector pIC (not drawn to scale). Since the NheI, SpeI and XbaI restrcition sites have complimentary overhangs, any of the backbone elements (SV40 ori, kanamycin resistance and puc ori) can be easily taken out and swapped or removed. In addition there is a Multiple Cloning Site (MCS) that contains unique sites, include a blunt cutter (EcoRV), for ease of insertion of heterologous sequences. The WPRE is included as a feature that enhances vector titer and gene expression, and is flanked by ScaI and EcoRV sites. The SV40 poly(A) enhancer element (box between BspE1-1807 and Agel-1853) is flanked by BspEI and AgeI sites that have complimentary overhangs. Extra elements can be inserted into 3′ U3 at the unique BspE1 site.

### EXAMPLE 2

### Lentiviral Transfer Vector pIC: Fragments to Assemble

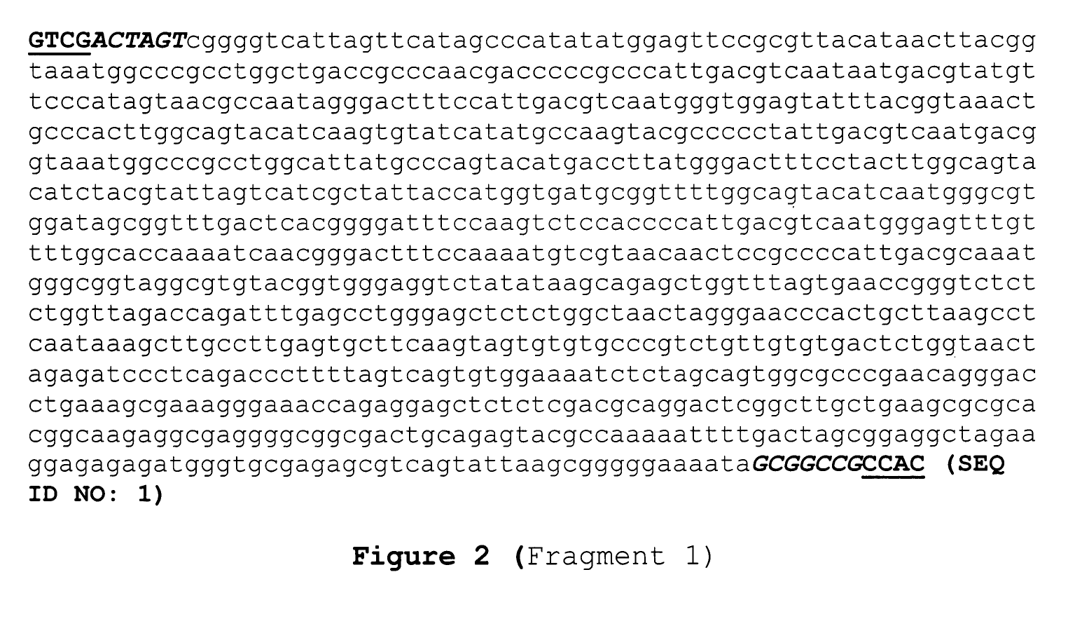

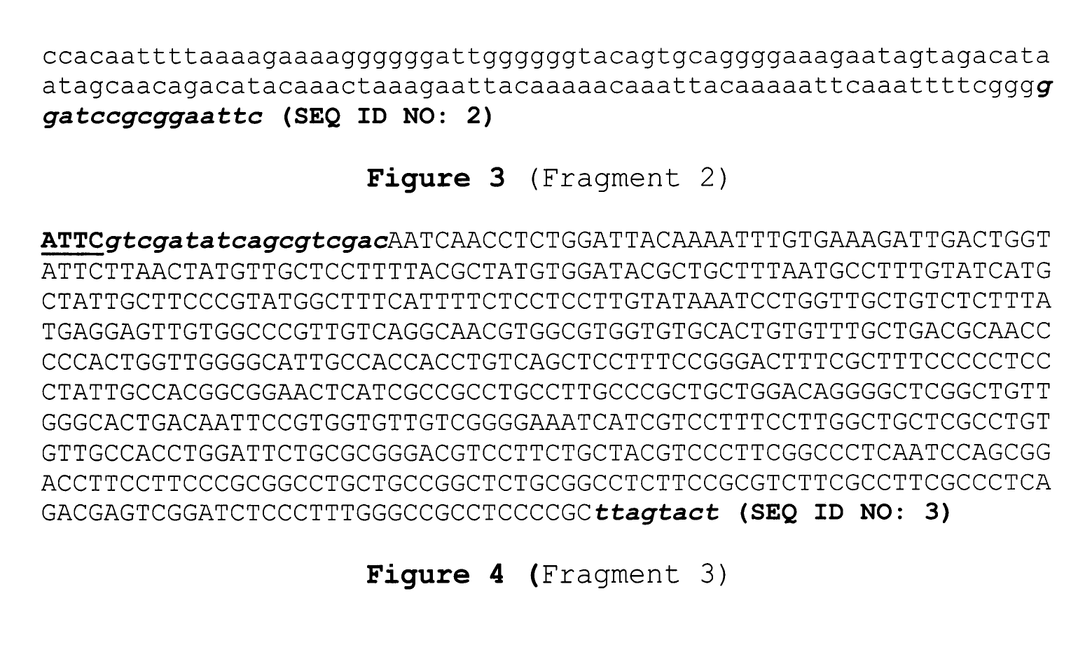

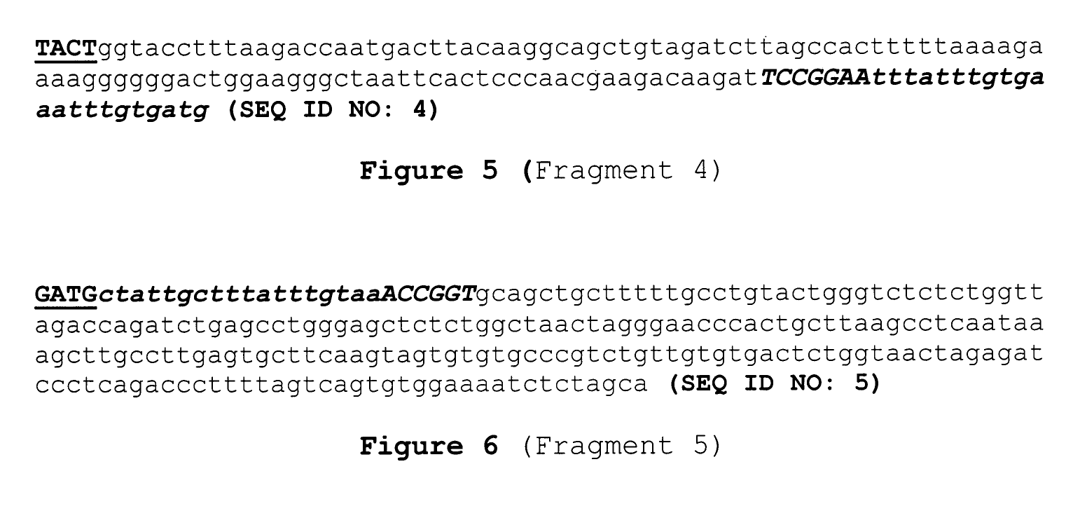

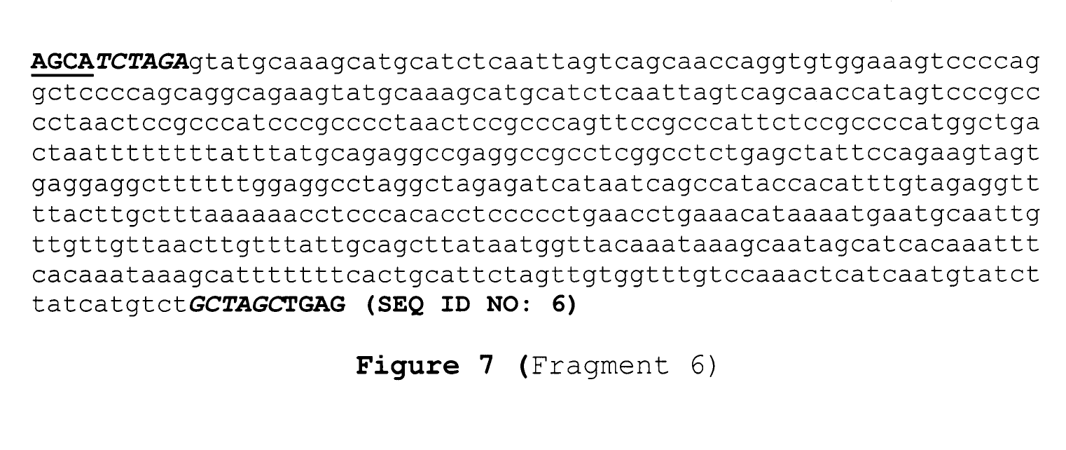

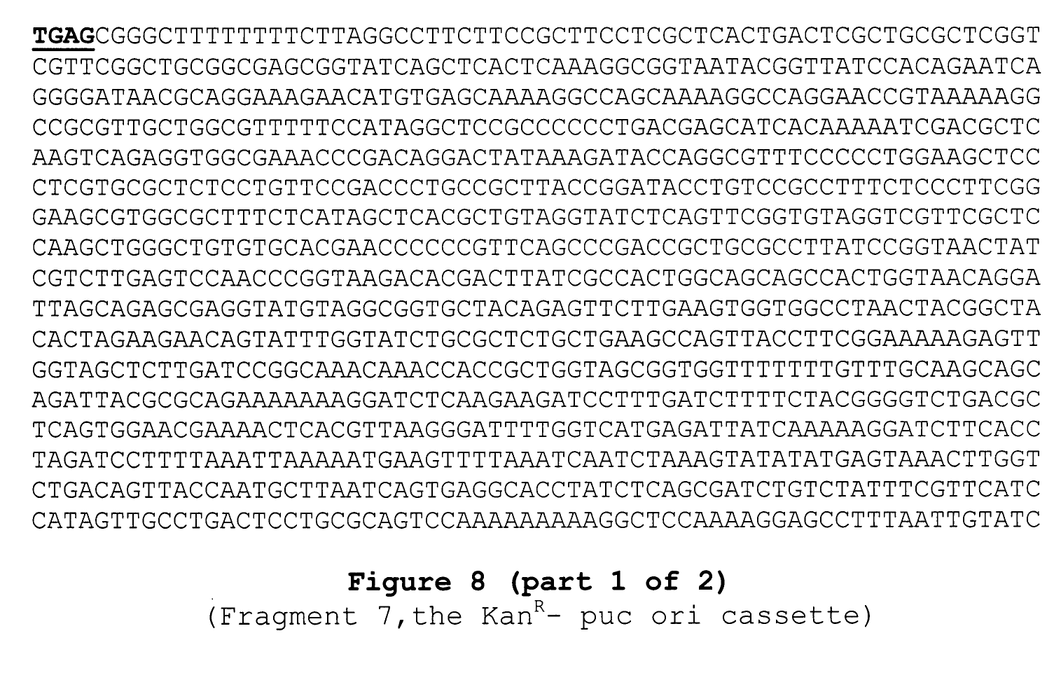

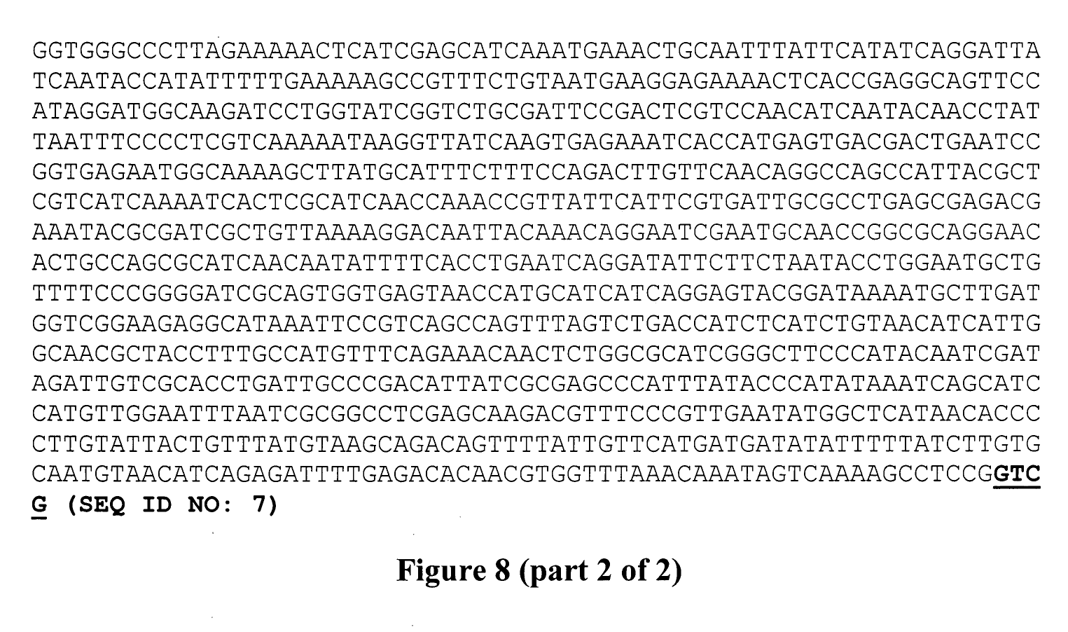

The lentiviral transfer vector pIC was assembled by assembling various fragments, designated fragments 1-7 in FIGS. 2-8 herein (SEQ ID NO: 1; SEQ ID NO: 2; SEQ ID NO: 3; SEQ ID NO: 4; SEQ ID NO: 5; SEQ ID NO: 6; SEQ ID NO: 7), using the primers shown below. In FIGS. 2-8, the sequences in normal font or italic font are the actual sequence present in the final plasmid construct. The sequences in underlined font overlap 4 bp stretches at the end of each fragment and are used to facilitate ligation of the fragments, but are not duplicated in the final products. Fragments were assembled using gene self-assembly (GENSA) technology (Solaiman F, et al., Modular retro-vectors for transgenic and therapeutic use. Mol Reprod Dev. 2000 June; 56(2 Suppl):309-315).

The fragments were assembled by polymerase chain reaction (PCR) in accordance with known techniques. The primers used for assembly of the fragments were as follows:

(SEQ ID NO: 8)

F1S: GTCGACTAGTcggggtcattagttcatagcccata

(SEQ ID NO: 9)

F1AS: gtggcggccgctattttcccccgcttaatactgac

(SEQ ID NO: 10)

F2S: ccacaattttaaaagaaaaggggggattg

(SEQ ID NO: 11)

F2AS: gaattccgcggatcccccgaaaatttgaatttttgtaatttg

(SEQ ID NO: 12)

F3S: ATTCgtcgatatcagcgtcgacAATCAACCTCTGGATTACAAAAT
TTGTG

(SEQ ID NO: 13)

F3AS: agtactaagcggggaggcggcccaaagggagatc

(SEQ ID NO: 14)

F4S: TACTggtacctttaagaccaatgacttac

(SEQ ID NO: 15)

F4AS: catcacaaatttcacaaataaattccggaatcttgtcttcgttg
ggagtgaat

(SEQ ID NO: 16)

F5S: GATGctattgctttatttgtaaACCGGTgcagctgctttttgcct
gtactgggt

(SEQ ID NO: 17)

F5AS: tgctagagattttccacactgactaaaag

(SEQ ID NO: 18)

F6S: AGCATCTAGAgtatgcaaagcatgcatctcaattagtc

(SEQ ID NO: 19)

F6AS: ctcagctagcagacatgataagatacattgatgagt

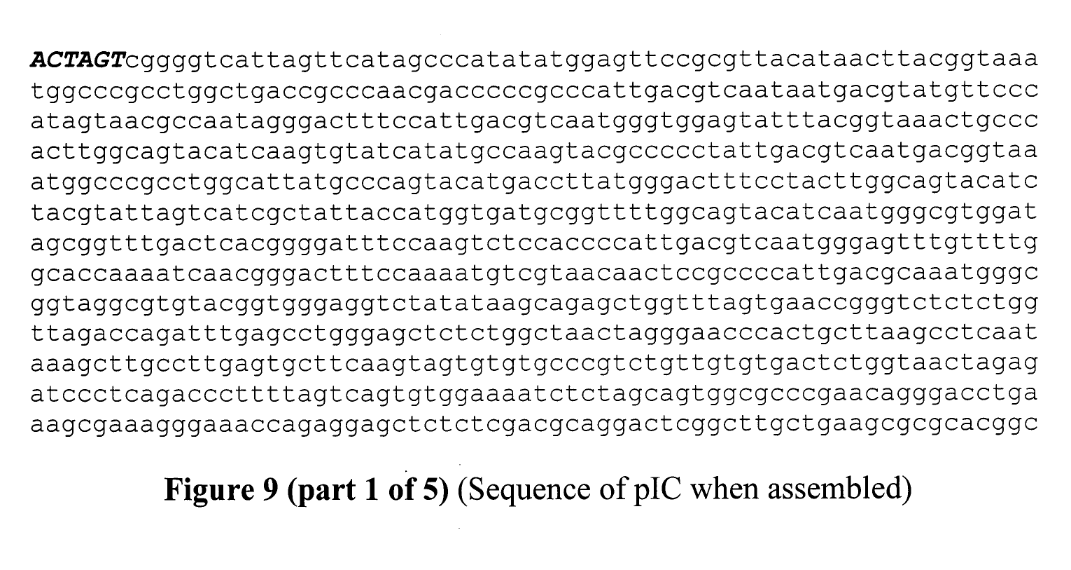

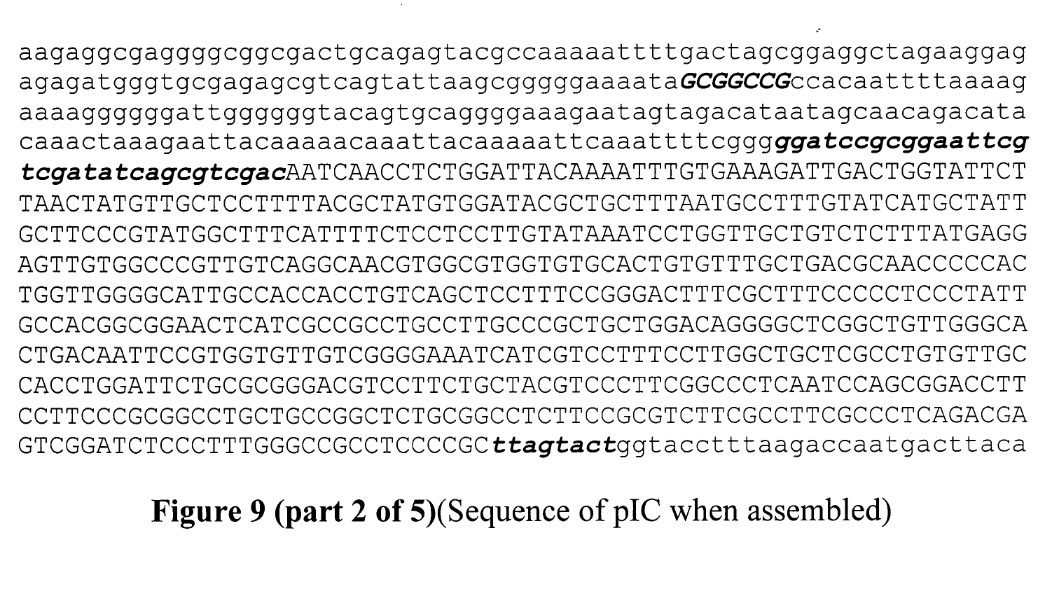

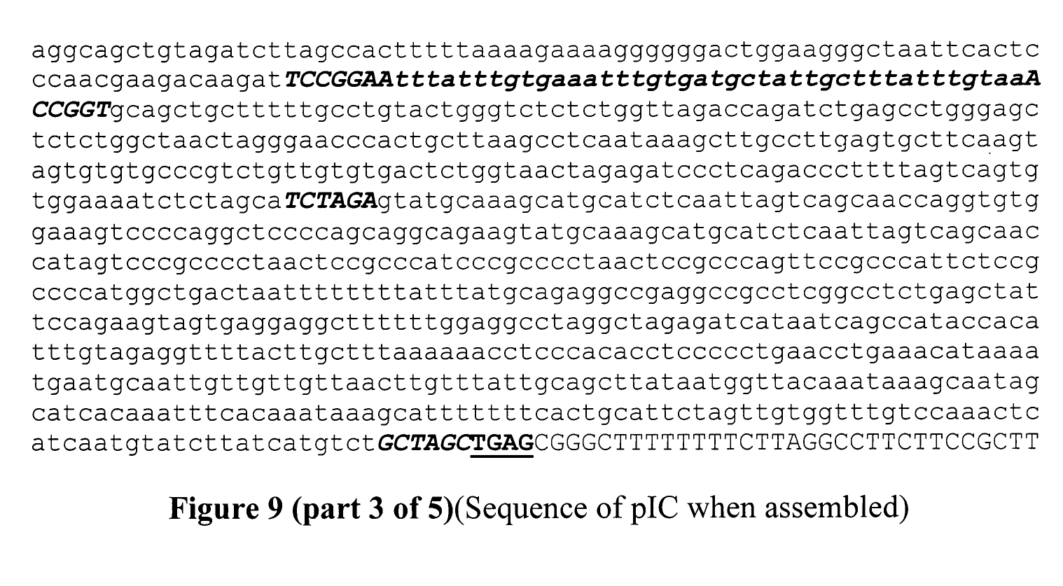

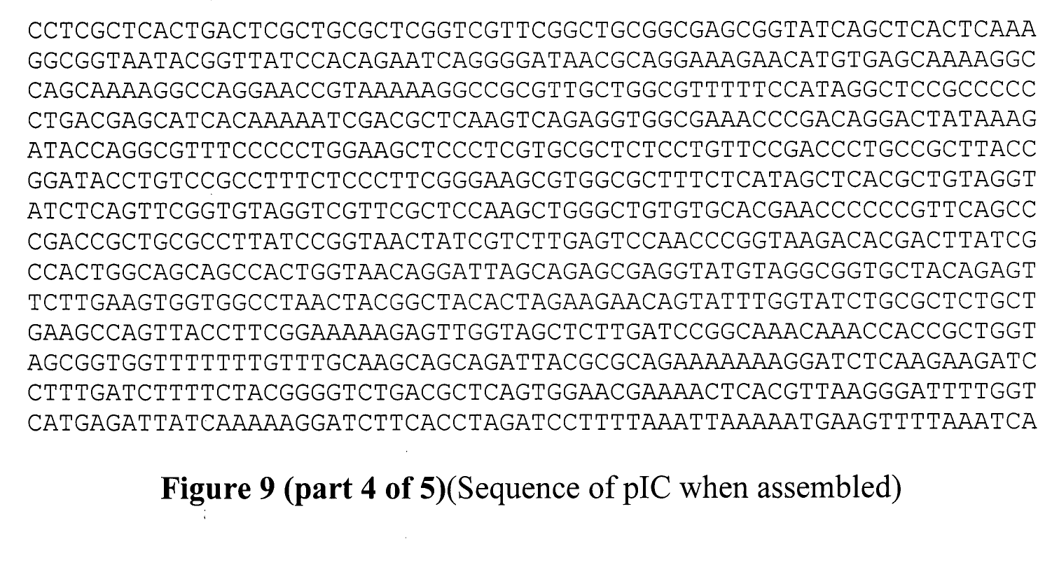

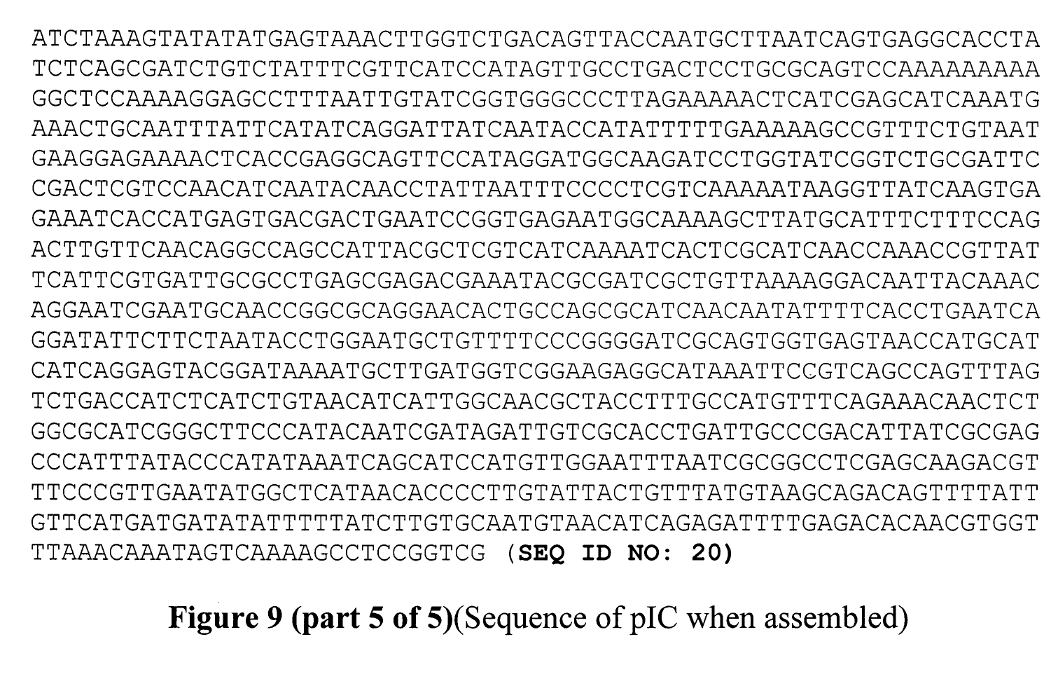

The final sequence of pIC, when assembled, was 4,637 bp, and is shown in FIG. 9 (SEQ ID NO: 20).

### EXAMPLE 3

### Construction of Packaging Construct, pGPRcwVA

(1) pGPR: expresses HIV-1 Gag-Pol from a CMV immediate early promoter, but does not contain any other HIV-1 genes (ie Env, Tat, Rev and the accessory genes Vpu, Vif, Vpr and Nef). It was derived from plasmid pHIV-DY, where the LTRs have been replaced by a CMV promoter/β-globin intron at the 5′ end, and a β-globin poly(A) sequence at the 3′ end, part of the env gene is deleted, and all the accessory proteins are retained (reference Sutton et al. 1998). We cut pHIV-DY with restriction sites PflM1 and Not I, and replaced this fragment with a PCR generated fragment corresponding to the HIV-1 Rev-response-element (RRE). The RRE fragment was generated by PCR using HIV-DY as a template. The sequence amplified was from 5592 to 5828 (numbering according to HIV-DY sequence). Restriction sites PflM1 and NotI were tagged to the 5′ and 3′ primers respectively. The RRE PCR fragment was cut with PflM1 and NotI restriction enzymes and cloned into the PflM1 and Not I cut HIV-DY plasmid. This resulted in plasmid pGPR.

(2) pGPRcw: The NheI-SphI fragment of plasmid pGPR was replaced with a fragment generated by splice-overlap PCR technique, and which differed from the pGPR sequence in the region corresponding to the first 40 bp of the Gag open reading frame. The splice overlap PCR fragment cut was NheI and SphI and cloned into pGPR, to create pGPRcw.

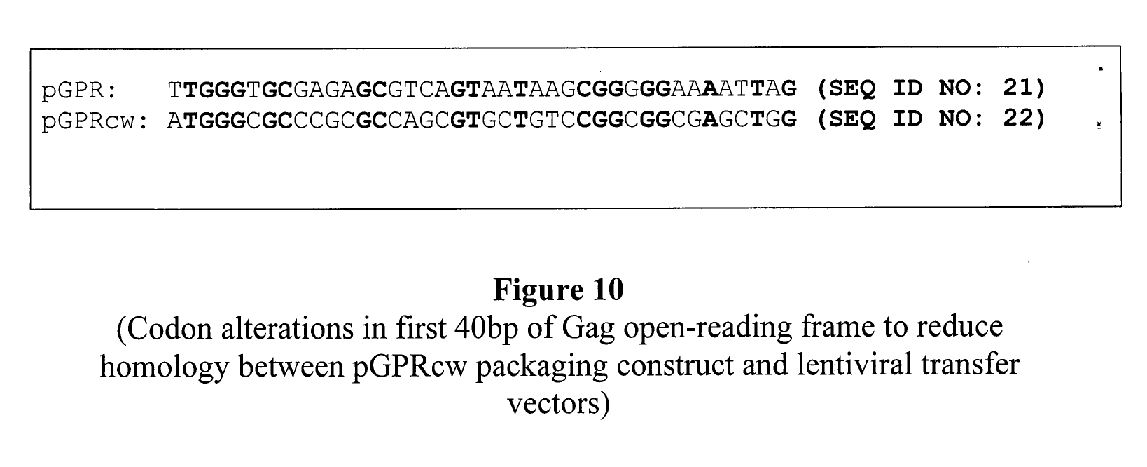

The sequence in this region was altered to contain codons optimized for human codon usage (FIG. 10) (SEQ ID NO: 21; SEQ ID NO: 22), which reduced the homology between the pGPRcw packaging construct and the lentiviral transfer vectors. In addition, the use of optimized human codons boosts production of viral particles in the producer cell line (Table 1).

(3) pGPRcwVA. Either VAI, or the whole VAI-II fragments were PCR'd from a template with the addition of NotI restriction sites at either end. The PCR products were digested and cloned into the single NotI site in pGPRcw, to create pGPRcwVAI and pGPRcwVA, respectively. The addition of either fragment boosted both vector titer (Table 1).

Primers used were:

(SEQ ID NO: 23)

VA-F (forward primer for both PCRs):
ccacgttgcggccgcgggcactcttccgtggtctggtgg

(SEQ ID NO: 24)

VAI-R (reverse primer for VAI):
gcacgagcggccgccaaaaggagcgctcccccgttgtctg

(SEQ ID NO: 25)

VAI-II-R (reverse primer for VAI-II):
gcacgagcggccgc aaaaaaggggctcgtccctgtttccgg

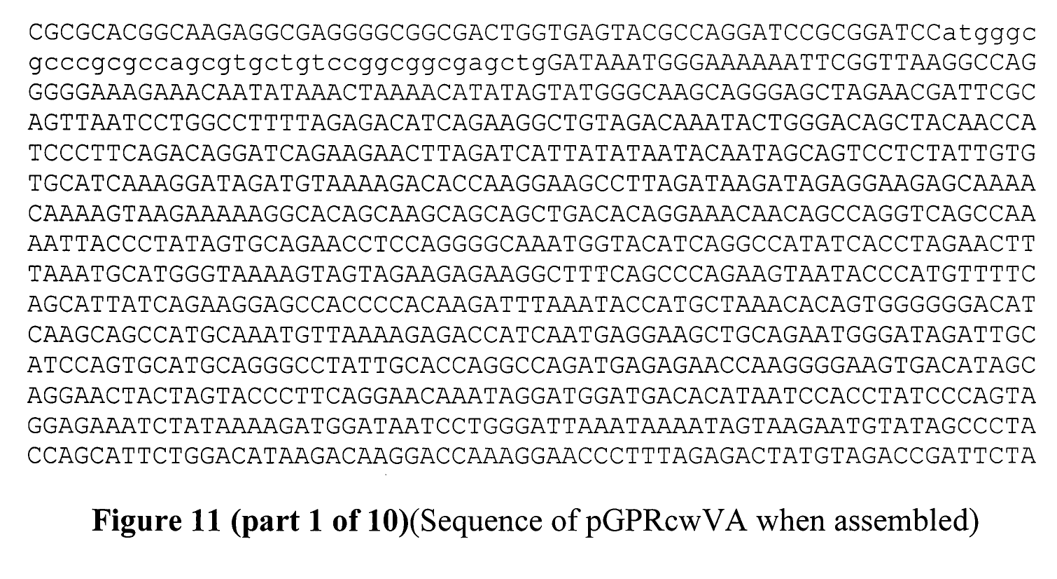

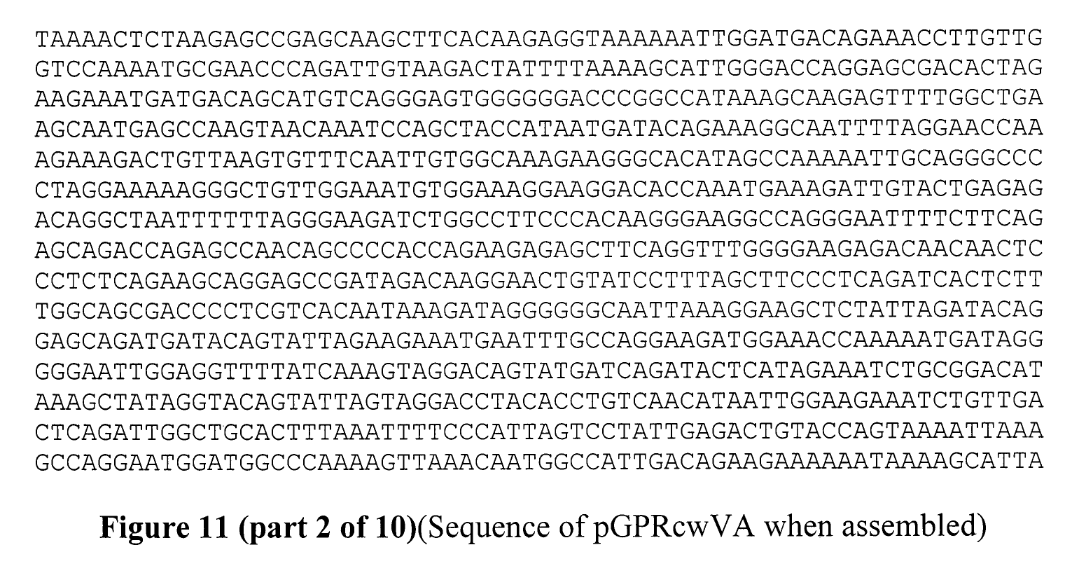

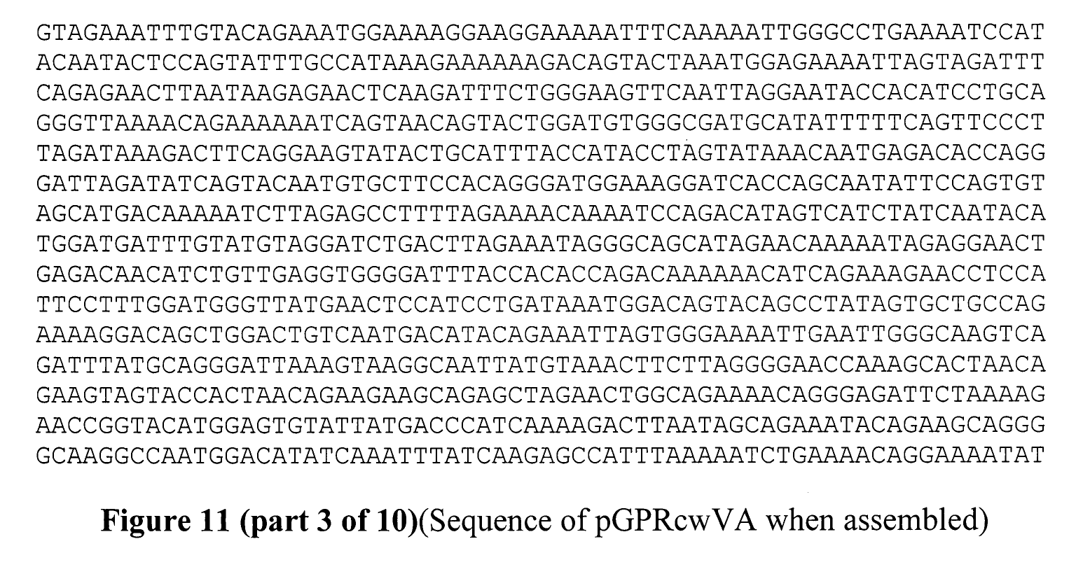

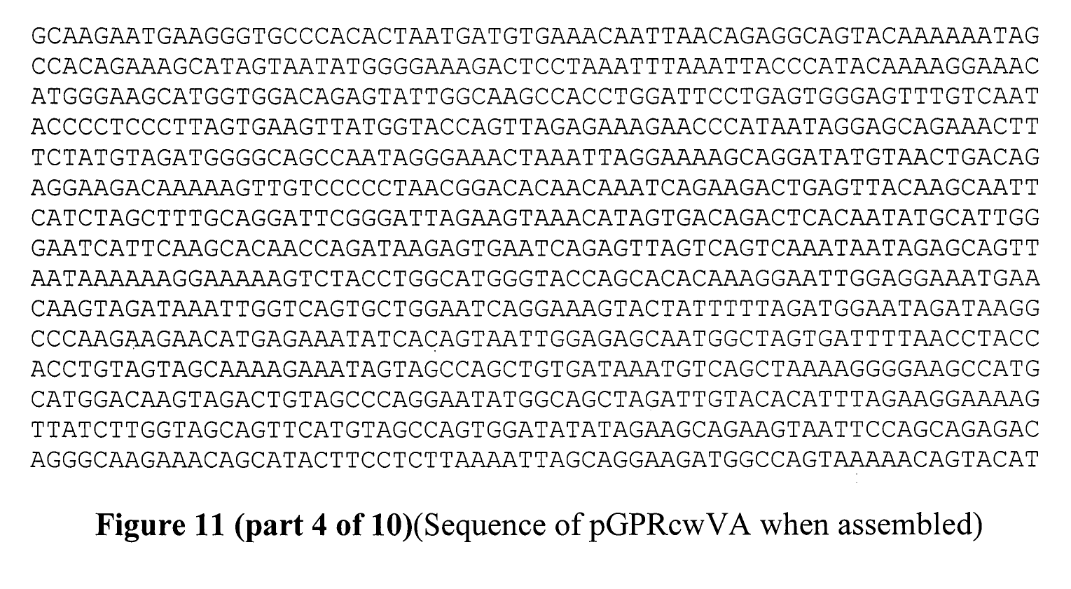

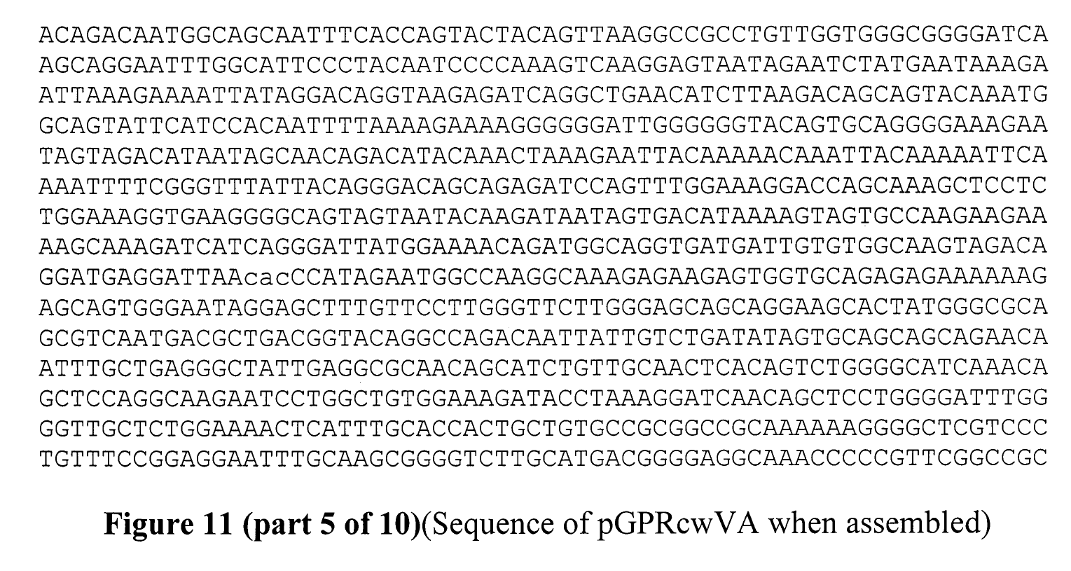

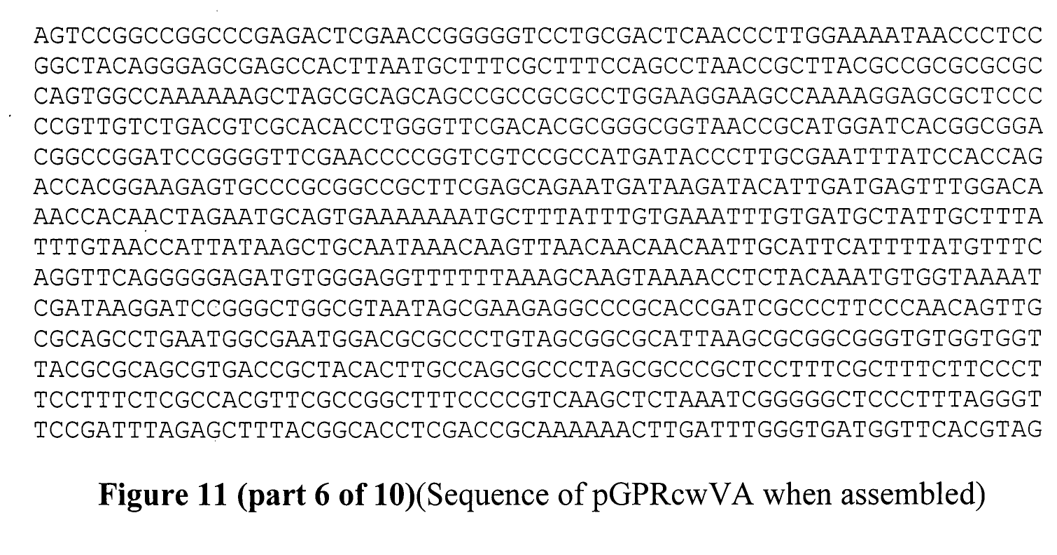

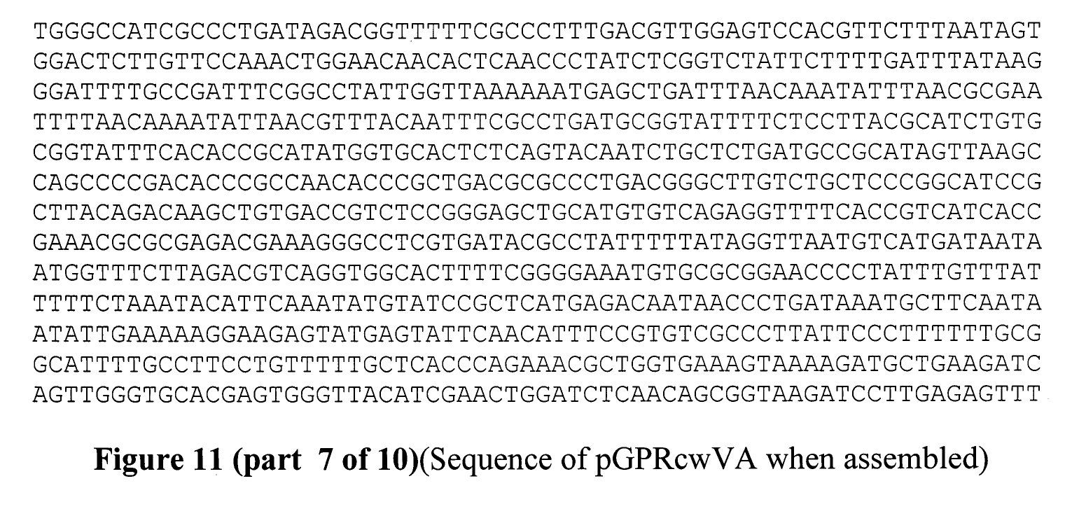

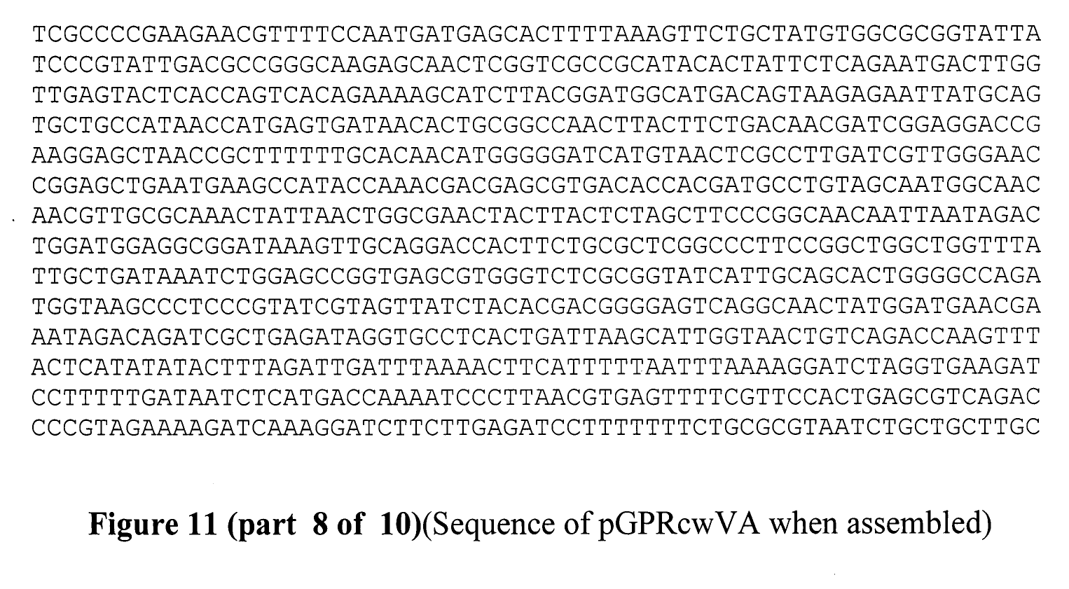

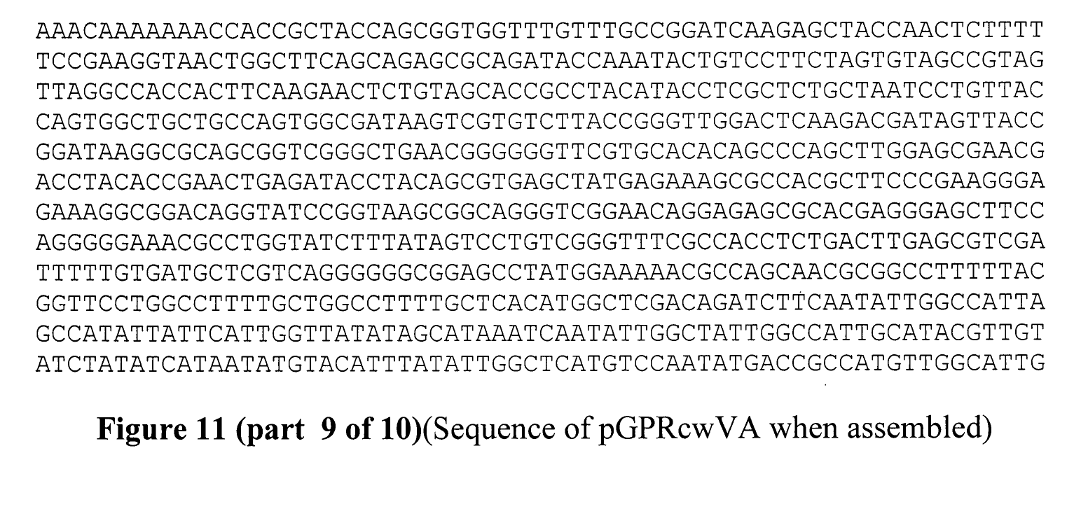

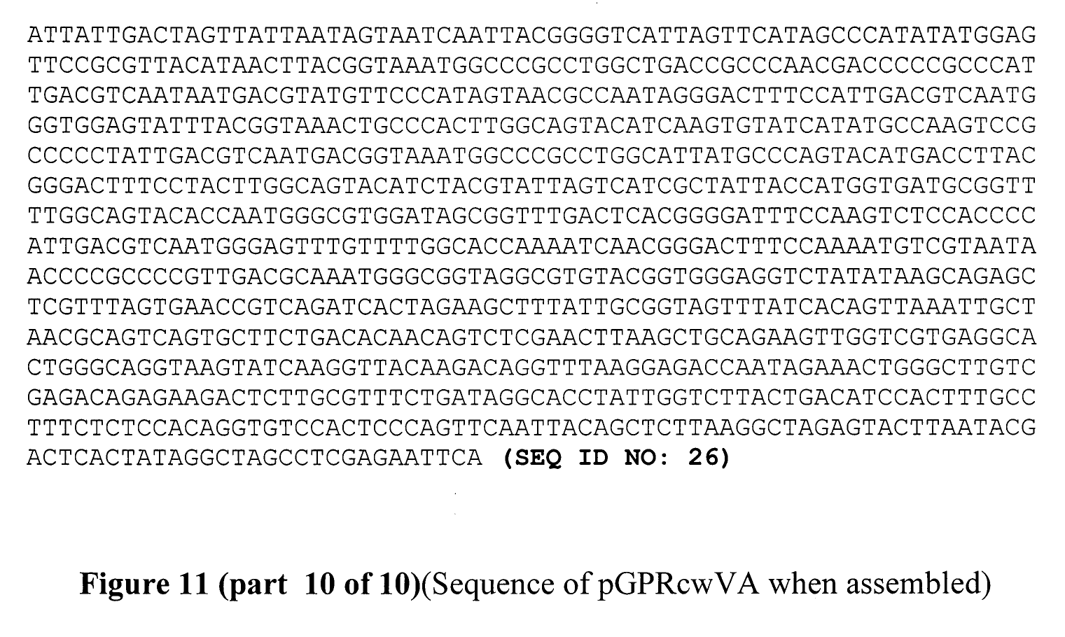

The final sequence of the finished construct is given in FIG. 11 (SEQ ID NO: 26).

See Sutton R E, Wu H T, Rigg R, Bohnlein E, Brown P O. 1998. Human immunodeficiency virus type 1 vectors efficiently transduce human hematopoietic stem cells. J. Virol. 72:5781-8.

### EXAMPLE 4

### Production of Viral Particles from Producer Cells

The various packaging constructs indicated were used to produce lentiviral vectors by transient transfection into 293T cells, together with a plasmid expressing VSV-G, a plasmid expressing Rev (where required) and the lentiviral transfer vector SMPU-CMV-LacZ. The titers of the lentiviral vectors so generated were measured on 293T cells, and made relative in each independent experiment to the titer obtained with the control standard packaging constructs, pHIV-DY or pCMVΔR8.2. In addition, the amount of viral particles in each vector preparation were measured using either a reverse transcriptase assay (Expt. 1) or p24 ELISA (Expt. 2). Data are given in Table 1 below.

TABLE 1

| | | | Particle production | |
|---|---|---|---|---|
| | | | RT activity | p24 levels |
| Packaging | Titer | | (arbitary units) | (ng/ml) |
| construct | Expt 1. | Expt 2. | Expt 1. | Expt 2. |
| pHIV-DY | 100 | | 10,283 | |
| pCMVΔR8.2 | | 100 | | |
| pGPR | 0.4 | | 1,207 | |
| pGPRcw | 6 | 36 | 2,635 | 2.6 |
| pGPRcwVA_{I} | 40 | | | 22.5 |
| pGPRcwVA_{I-II} | | 109 | | |

### EXAMPLE 5

### Effect on Titer of Presence of Various Elements

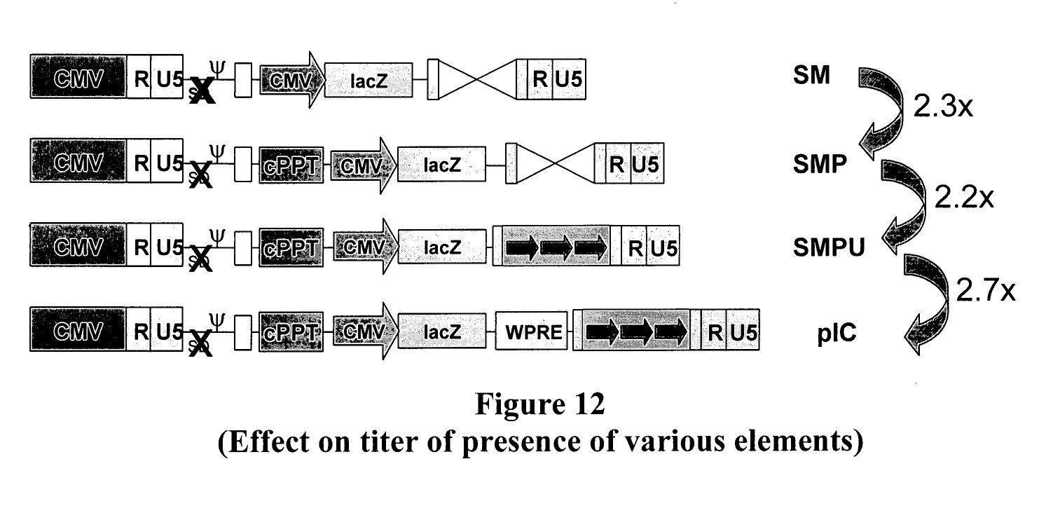

The effect on titer of the presence of various elements in the transfer vector is shown in FIG. 12. Lentiviral vectors were generated by transient transfection of 293T cells with the designated transfer vector, together with a plasmid expressing VSV-G, a plasmid expressing Rev and packaging construct pGPRcw. Addition of the cPPT (vector SMP) increases titer by 2.3-fold, the upstream enhancer (SMPU) increases titer by a further 2.2-fold, and addition of the WPRE (PIC) increased titer a further 2.7-fold. Vector pIC results in the highest titers.

The foregoing is illustrative of the present invention, and is not to be construed as limiting thereof. The invention is defined by the following claims, with equivalents of the claims to be included therein.

That which is claimed is:

1. A lentiviral transfer vector comprising:
a) a 5′ LTR;
b) a 3′ LTR comprising a polyadenylation signal;
c) a minimal packaging signal,
d) (i) at least one heterologous upstream enhancer (UE) sequences, and/or (ii) at least one additional copy of endogenous UE sequences operatively associated with said polyadenylation signal; and
e) a PRE.

2. The lentiviral vector of claim 1, wherein said PRE is a WPRE.

3. The vector of claim 1, further comprising:
f) a cPPT.

4. The vector of claim 1 which comprises several heterologous UE sequences at least two of which are derived from the same UE

5. The vector of claim 1 which comprises:
several heterologous UE sequences at least two of which are derived from different UEs;
several additional copies of endogenous UE sequences at least two of which copies have identical sequences; and/or
several additional copies of endogenous UE sequences at least two of which copies have different sequences.

6. The vector of claim 1, wherein said 3′ LTR comprises a deletion of U3 promoter sequence.

7. The vector of claim 6, wherein said heterologous UE sequence, or at least one of said heterologous UE sequences, or said additional copy of endogenous UE sequence, or at least one of said additional copies of endogenous sequences is at the U3 promoter deletion site.

8. The vector of claim 1, wherein said heterologous UE sequence or at least one of said heterologous UE sequences is a viral or animal UE sequence.

9. The vector of claim 1, wherein said 3′ LTR comprises an endogenous UE sequence.

10. The vector of claim 1, wherein said 5′ LTR comprises a heterologous promoter inserted in the U3 region.

11. The vector of claim 10, wherein 5′ LTR comprises a deletion of the endogenous U3 promoter sequence.

12. The vector of claim 1, which comprises a heterologous coding sequence which is 3′ downstream of said 5′ LTR and 5′ upstream of said 3′ LTR.

13. The vector of claim 12, wherein said heterologous coding sequence encodes a protein, peptide or RNA.

14. The vector of claim 12, wherein said heterologous coding sequence encodes a negative selective marker.

15. The vector of claim 1 having the nucleic acid sequence given herein as SEQ ID NO: 20, subject to the proviso that a heterologous coding sequence may optionally be inserted therein 3′ downstream of the vector 5′ LTR and 5′ upstream of the vector 3′ LTR.

16. A cell comprising the vector of claim 1.

17. The cell of claim 16 which is a mammalian cell.

18. The cell of claim 17 which is a human cell.

19. A cell comprising a proviral sequence transcribed from the vector of claim 1.

20. The cell of claim 19 which is a mammalian cell.

21. The cell of claim 20 which is a human cell.

22. An infectious lentiviral particle produced by the cell of claim 19, wherein said cell is a producer cell.

23. A method for expressing a heterologous coding sequence in a cell comprising delivering to said cell the vector of claim 12.

24. The method according to claim 23, wherein said heterologous coding sequence encodes a protein, peptide or RNA.

25. The method according to claim 23, wherein said heterologous coding sequence encodes a negative selective marker.

26. A method for expressing a heterologous coding sequence in a cell comprising delivering to said cell an infectious viral particle comprising the vector of claim 12.

27. The method according to claim 26, wherein said heterologous coding sequence encodes a protein, peptide or RNA.

28. The method according to claim 26, wherein said heterologous coding sequence encodes a negative selective marker.

29. A method for expressing a heterologous coding sequence in a cell of a subject comprising delivering to said host an infectious retroviral particle comprising the vector of claim 12.

30. The method according to claim 29, wherein said heterologous coding sequence encodes a protein, peptide or RNA.

31. The method according to claim 29, wherein said heterologous coding sequence encodes a negative selective marker.

32. The method according to claim 29, wherein said subject is a mammal.

33. The method according to claim 29, wherein said subject is a human.

34. A method for expressing a heterologous coding sequence in a subject comprising transfusing said subject with a composition comprising the cell of claim 19.

35. The method according to claim 34, wherein said subject is a mammal.

36. The method according to claim 34, wherein said subject is a human.

37. A packaging construct comprising a nucleic acid encoding and expressing a lentiviral Gag nucleic acid; wherein said lentiviral Gag nucleic acid is a mutated Gag nucleic acid containing one or more substitution mutations, wherein said mutated Gag nucleic acid encodes the same amino acid sequence as the corresponding unmutated Gag nucleic acid, but differs from the nucleic acid sequence of said corresponding unmutated Gag nucleic acid sequence due to the degeneracy of the genetic code.

38. The packaging construct of claim 37 encoding and expressing lentiviral Gag and Pol nucleic acid.

39. The packaging construct of claim 37, wherein the first 30 5′ nucleic acids of said mutated Gag nucleic acid do not contain more than 12 consecutive unmutated nucleic acid residues.

40. The packaging construct of claim 37, further comprising a heterologous nucleic acid encoding and expressing an adenovirus VA RNA.

41. The packaging construct of claim 40, wherein said adenovirus VA RNA is adenovirus VA1 RNA, adenovirus VA2 RNA, or combinations thereof.

42. The packaging construct of claim 37 having the sequence given herein as SEQ ID NO: 26.

43. A lentiviral vector producer cell comprising a heterologous nucleic acid encoding and expressing a lentiviral Gag nucleic acid; wherein said lentiviral Gag nucleic acid is a mutated Gag nucleic acid containing one or more substitution mutations, wherein said mutated Gag nucleic acid encodes the same amino acid sequence as the corresponding unmutated Gag nucleic acid, but differs from the nucleic acid sequence of said corresponding unmutated Gag nucleic acid sequence due to the degeneracy of the genetic code.

44. The producer cell of claim 43, said heterologous nucleic acid encoding and expressing lentiviral Gag and Pol nucleic acid.

45. The producer cell of claim 43, wherein the first 30 5′ nucleic acids of said mutated Gag nucleic acid do not contain more than 12 consecutive unmutated nucleic acid residues.

46. The producer cell of claim 43, wherein said Gag is constitutively, inducibly or transiently expressed.

47. The producer cell according to claim 43, further comprising a heterologous nucleic acid encoding and expressing an adenovirus VA RNA.

48. The producer cell according to claim 43, wherein said adenovirus VA RNA is adenovirus VA1 RNA, adenovirus VA2 RNA, or combinations thereof.

49. The producer cell of claim 48, wherein said adenovirus VA RNA is constitutitively, inducibly or transiently expressed.

50. A method of making lentiviral particles comprising the steps of:
(a) providing a lentiviral vector producer cell comprising a heterologous nucleic acid encoding and expressing a lentiviral Gag nucleic acid; wherein said lentiviral Gag nucleic acid is a mutated Gag nucleic acid containing one or more substitution mutations, wherein said mutated Gag nucleic acid encodes the same amino acid sequence as the corresponding unmutated Gag nucleic acid, but differs from the nucleic acid sequence of said corresponding unmutated Gag nucleic acid sequence due to the degeneracy of the genetic code;
(b) introducing the lentiviral vector of claim 1 into said producer cell, said lentiviral vector further comprising said corresponding umnutated Gag nucleic acid; and then
(c) collecting lentiviral particles from said producer cells.

51. The method of claim 50, wherein said producer cell is a mammalian cell.

52. The method of claim 50, said heterologous nucleic acid in said producer cell encoding and expressing lentiviral Gag and Pol nucleic acid.

53. A packaging construct comprising:
a nucleic acid encoding and expressing a lentiviral Gag nucleic acid; and
a heterologous nucleic acid encoding and expressing an adenovirus VA RNA.

54. The packaging construct of claim 53, said nucleic acid encoding and expressing lentiviral Gag and Pol nucleic acid.

55. The packaging construct of claim 53, wherein said adenovirus VA RNA is adenovirus VA1 RNA, adenovirus VA2 RNA, or combinations thereof.

56. A lentiviral vector producer cell comprising:
a heterologous nucleic acid encoding and expressing a lentiviral Gag nucleic acid; and
a heterologous nucleic acid encoding and expressing an adenovirus VA RNA.

57. The producer cell of claim 56, said heterologous nucleic acid encoding and expressing lentiviral Gag and Pol nucleic acid.

58. The producer cell of claim 56, wherein said Gag is constitutively, inducibly or transiently expressed.

59. The producer cell according to claim 56, wherein said adenovirus VA RNA is adenovirus VA1 RNA, adenovirus VA2 RNA, or combinations thereof.

60. The producer cell of claim 56, wherein said adenovirus VA RNA is constitutitively, inducibly or transiently expressed.

61. A method of making lentiviral particles comprising the steps of:
(a) providing a lentiviral vector producer cell comprising a heterologous nucleic acid encoding and expressing a lentiviral Gag nucleic acid, and a heterologous nucleic acid encoding and expressing an adenovirus VA RNA;
(b) introducing the lentiviral vector of claim 1 into said producer cell; and then
(c) collecting lentiviral particles from said producer cells.

62. The method of claim 61, wherein said producer cell is a mammalian cell.

63. The method of claim 61, said heterologous nucleic acid in said producer cell encoding and expressing lentiviral Gag and Pol nucleic acid.


SEQUENCE LISTING (26 sequences)

SEQ ID NO: 1 - DNA, 953 bp, Artificial
  PCR product
gtcgactagtcggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgggtctctctggttagaccagatttgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactgcagagtacgccaaaaattttgactagcggaggctagaaggagagagatgggtgcgagagcgtcagtattaagcgggggaaaatagcggccgccac

SEQ ID NO: 2 - DNA, 142 bp, Artificial
  PCR product
ccacaattttaaaagaaaaggggggattggggggtacagtgcaggggaaagaatagtagacataatagcaacagacatacaaactaaagaattacaaaaacaaattacaaaaattcaaattttcgggggatccgcggaattc

SEQ ID NO: 3 - DNA, 619 bp, Artificial
  PCR product
attcgtcgatatcagcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcttagtact

SEQ ID NO: 4 - DNA, 139 bp, Artificial
  PCR product
tactggtacctttaagaccaatgacttacaaggcagctgtagatcttagccactttttaaaagaaaaggggggactggaagggctaattcactcccaacgaagacaagattccggaatttatttgtgaaatttgtgatg

SEQ ID NO: 5 - DNA, 231 bp, Artificial
  PCR product
gatgctattgctttatttgtaaaccggtgcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 6 - DNA, 532 bp, Artificial
  PCR product
agcatctagagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggctagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgctagctgag

SEQ ID NO: 7 - DNA, 2049 bp, Artificial
  Plasmid cassette sequence
tgagcgggcttttttttcttaggccttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcgcagtccaaaaaaaaaggctccaaaaggagcctttaattgtatcggtgggcccttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggtttaaacaaatagtcaaaagcctccggtcg

SEQ ID NO: 8 - DNA, 35 bp, Artificial
  PCR primer
gtcgactagtcggggtcattagttcatagcccata

SEQ ID NO: 9 - DNA, 35 bp, Artificial
  PCR primer
gtggcggccgctattttcccccgcttaatactgac

SEQ ID NO: 10 - DNA, 29 bp, Artificial
  PCR primer
ccacaattttaaaagaaaaggggggattg

SEQ ID NO: 11 - DNA, 42 bp, Artificial
  PCR primer
gaattccgcggatcccccgaaaatttgaatttttgtaatttg

SEQ ID NO: 12 - DNA, 50 bp, Artificial
  PCR primer
attcgtcgatatcagcgtcgacaatcaacctctggattacaaaatttgtg

SEQ ID NO: 13 - DNA, 34 bp, Artificial
  PCR primer
agtactaagcggggaggcggcccaaagggagatc

SEQ ID NO: 14 - DNA, 29 bp, Artificial
  PCR primer
tactggtacctttaagaccaatgacttac

SEQ ID NO: 15 - DNA, 53 bp, Artificial
  PCR primer
catcacaaatttcacaaataaattccggaatcttgtcttcgttgggagtgaat

SEQ ID NO: 16 - DNA, 54 bp, Artificial
  PCR primer
gatgctattgctttatttgtaaaccggtgcagctgctttttgcctgtactgggt

SEQ ID NO: 17 - DNA, 29 bp, Artificial
  PCR primer
tgctagagattttccacactgactaaaag

SEQ ID NO: 18 - DNA, 38 bp, Artificial
  PCR primer
agcatctagagtatgcaaagcatgcatctcaattagtc

SEQ ID NO: 19 - DNA, 36 bp, Artificial
  PCR primer
ctcagctagcagacatgataagatacattgatgagt

SEQ ID NO: 20 - DNA, 4637 bp, Artificial
  pIC complete plasmid sequence
actagtcggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgggtctctctggttagaccagatttgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactgcagagtacgccaaaaattttgactagcggaggctagaaggagagagatgggtgcgagagcgtcagtattaagcgggggaaaatagcggccgccacaattttaaaagaaaaggggggattggggggtacagtgcaggggaaagaatagtagacataatagcaacagacatacaaactaaagaattacaaaaacaaattacaaaaattcaaattttcgggggatccgcggaattcgtcgatatcagcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcttagtactggtacctttaagaccaatgacttacaaggcagctgtagatcttagccactttttaaaagaaaaggggggactggaagggctaattcactcccaacgaagacaagattccggaatttatttgtgaaatttgtgatgctattgctttatttgtaaaccggtgcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcatctagagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggctagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgctagctgagcgggcttttttttcttaggccttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcgcagtccaaaaaaaaaggctccaaaaggagcctttaattgtatcggtgggcccttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggtttaaacaaatagtcaaaagcctccggtcg

SEQ ID NO: 21 - DNA, 40 bp, Artificial
  pGPR sequence optimized for human codon usage
ttgggtgcgagagcgtcagtaataagcgggggaaaattag

SEQ ID NO: 22 - DNA, 40 bp, Artificial
  pGPRcw sequence optimized for human codon usage
atgggcgcccgcgccagcgtgctgtccggcggcgagctgg

SEQ ID NO: 23 - DNA, 39 bp, Artificial
  PCR primer
ccacgttgcggccgcgggcactcttccgtggtctggtgg

SEQ ID NO: 24 - DNA, 40 bp, Artificial
  PCR primer
gcacgagcggccgccaaaaggagcgctcccccgttgtctg

SEQ ID NO: 25 - DNA, 41 bp, Artificial
  PCR primer
gcacgagcggccgcaaaaaaggggctcgtccctgtttccgg

SEQ ID NO: 26 - DNA, 9117 bp, Artificial
  pGPRcwVA complete plasmid sequence
cgcgcacggcaagaggcgaggggcggcgactggtgagtacgccaggatccgcggatccatgggcgcccgcgccagcgtgctgtccggcggcgagctggataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaatgcatgggtaaaagtagtagaagagaaggctttcagcccagaagtaatacccatgttttcagcattatcagaaggagccaccccacaagatttaaataccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaggacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaaatatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacccatagaatggccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctgatatagtgcagcagcagaacaatttgctgagggctattgaggcgcaacagcatctgttgcaactcacagtctggggcatcaaacagctccaggcaagaatcctggctgtggaaagatacctaaaggatcaacagctcctggggatttggggttgctctggaaaactcatttgcaccactgctgtgccgcggccgcaaaaaaggggctcgtccctgtttccggaggaatttgcaagcggggtcttgcatgacggggaggcaaacccccgttcggccgcagtccggccggcccgagactcgaaccgggggtcctgcgactcaacccttggaaaataaccctccggctacagggagcgagccacttaatgctttcgctttccagcctaaccgcttacgccgcgcgcgccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcggccgcttcgagcagaatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattca